(19) United States
(12) Patent Application Publication
(10) Pub. No.: US 2014/0051593 A1
(43) Pub. Date: Feb. 20, 2014

(54) Assay Methods and Systems

(71) Applicant: NVS Technologies, Inc., Menlo Park, CA (US)

(72) Inventors: Brad Taft, San Francisco, CA (US); Kris Scaboo, Castro Valley, CA (US); Jason La, San Francisco, CA (US); Christine Hardy, San Francisco, CA (US)

(21) Appl. No.: 13/844,426

(22) Filed: Mar. 15, 2013

Related U.S. Application Data
(60) Provisional application No. 61/684,104, filed on Aug. 16, 2012.

(51) Int. Cl. C12Q 1/70

(52) U.S. Cl. CPC C12Q 1/701
USPC 506/9; 435/6.11; 435/5; 435/287.2; 506/16

(57) ABSTRACT

Assay methods and systems that detect and quantify target nucleic acid sequences in samples, employing amplification processes and real time amplification processes in the presence of target specific probe sequences and capture probe sequences for indication of the amplification of, and therefor the presence of the target sequence in the sample.

### CROSS-REFERENCE TO RELATED APPLICATIONS

This application claims priority to and benefit of U.S. provisional patent application Ser. No. 61/684,104, filed Aug. 16, 2012, the specification of which is incorporated herein in its entirety for all purposes.

### STATEMENT REGARDING FEDERALLY SPONSORED RESEARCH

This invention was made with support of a U.S. Dept. of Homeland Security grant, Contract Number HSHQDC-10-C-00053. The government has certain rights in the invention.

### BACKGROUND OF THE INVENTION

There have been developed a large number of different methods for detecting the presence of a given nucleic acid sequence in a sample mixture. These methods are exploited for purposes of diagnostics, research tools, pathogen detection, and many other applications. One method that has proven highly useful in identifying the presence of a given target nucleic acid sequence, particularly in situations where the copy number of such a target is expected to be low, is a real-time polymerase chain reaction, or real time PCR.

Real time PCR is routinely used for detection of nucleic acids of interest in a biological sample. For a review of real time PCR see, e.g., M Tevfik Dorak (Editor) (2006) Real-time PCR (Advanced Methods) Taylor & Francis, 1st edition ISBN-10: 041537734X ISBN-13: 978-0415377348, and Logan et al. (eds.) (2009) Real-Time PCR: Current Technology and Applications, Caister Academic Press, 1st edition ISBN-10: 1904455395, ISBN-13: 978-1904455394. For additional details, see also, e.g., Gelfand et al. “Homogeneous Assay System Using The Nuclease Activity of A Nucleic Acid Polymerase” U.S. Pat. No. 5,210,015; Leone et al. (1995) “Molecular beacon probes combined with amplification by NASBA enable homogenous real-time detection of RNA” Nucleic Acids Res. 26:2150-2155; and Tyagi and Kramer (1996) “Molecular beacons: probes that fluoresce upon hybridization” Nature Biotechnology 14:303-308. Traditionally, single well multiplexing, used to detect more than one target nucleic acid per sample in a single reaction container (e.g., well of a multiwell plate), is achieved using self-quenched PCR probes such as TAQMAN™ or molecular beacon probes that are specific for each amplicon. Upon binding to the amplicon in solution, or upon degradation of the probes during PCR, the probes unquench, producing a detectable signal. The probes are labeled with fluorophores of different wavelengths, permitting a multiplexing capability of up to about 5 targets in a single “one pot” reaction. More than about 5 probes per reaction is difficult to achieve, due to practical spectral range and label emission limitations. This severely limits multiplexing of a single reaction, which, in turn, significantly limits how many targets can be screened per sample and drives up reagent cost and instrument complexity in detecting multiple targets of interest.

Nucleic acid arrays represent another approach to multiplexing the detection of amplification products. Most typically, amplification reactions are performed on a sample, and amplicons are separately detected on a nucleic acid array. For example, Sorge “Methods for Detection of a Target Nucleic Acid Using A Probe Comprising Secondary Structure” U.S. Pat. No. 6,350,580 proposes the capture of a probe that is released upon amplification by purifying the probe out of the amplification mixture and then detecting it. This multiple-step approach to making and detecting amplicons makes real time analysis of the amplification mixture impractical.

Various approaches that amplify the reactants in the presence of capture nucleic acids have also been proposed. For example, Kleiber et al. “Integrated Method and System for Amplifying And Detecting Nucleic Acids,” U.S. Pat. No. 6,270,965, proposes detection of an amplicon via evanescence induced fluorescence. Similarly, Alexandre, et al. “Identification and Quantification of a Plurality of Biological (Micro) Organisms or Their Components,” U.S. Pat. No. 7,829,313, proposes detection of amplicons on arrays. In another example, target polynucleotides are detected by detecting a probe fragment that is produced as a result of amplification, e.g., by binding to an electrode, followed by electrochemical detection. See, e.g., Aivazachvilli et al. “Detection of Nucleic Acid Amplification” US Pub. No. 2007/0099211; Aivazachvilli et al. “Systems and Methods for Detecting Nucleic Acids US Pub. No. 2008/0193940, and Scaboo et al. “Methods And Systems for Detecting Nucleic Acids” US Pub. No. 2008/0241838.

These methods all suffer from practical limitations that limit their use for multiplex target nucleic acid detection. For example, Kleiber (U.S. Pat. No. 6,270,965) relies on evanescence induced fluorescence to detect fluorescence of amplicons at the array surface, and requires complex and expensive optics and arrays. Alexandre (U.S. Pat. No. 7,829,313) proposes detection of amplicons on an array; as in Kleiber this increases array costs significantly, because each array has to be custom designed to detect each amplicon. In practice, it can be difficult to achieve similar hybridization kinetics for disparate amplicons on an array, particularly where the amplicons are relatively large, as in Alexandre. Furthermore, this art provides little guidance regarding how to detect signal on an array where there is an accompanying solution phase that also comprises high levels of signal background, or of arrays that remain stable through in situ thermal cycling.

The present invention overcomes these and other problems in the art. A more complete understanding of the invention will be obtained upon complete review of the following.

### BRIEF SUMMARY OF THE INVENTION

The present invention provides novel assay methods and systems, as well as devices, reagents and reaction mixtures used in such methods and systems. In at least one aspect, the invention provides a method of detecting the presence of (and optionally quantifies an increase in amount of) at least a first target nucleic acid sequence in a sample. The methods comprise subjecting the sample to an amplification reaction capable of amplifying the target nucleic acid sequence(es) in the presence of at least a first set of nucleic acid probes, the first set of nucleic acid probes. The first set of probes comprises a capture probe comprising a fluorophore attached thereto, a target specific nucleic acid probe complementary to at least a portion of the capture probe and to at least a portion of a target nucleic acid sequence, and comprising a quencher attached thereto, such that the quencher quenches fluorescence from the fluorophore when the target specific probe is hybridized to the capture probe. The methods further comprise detecting fluorescence from the sample following one or more cycles of the polymerase chain reaction, an increase in fluorescence being indicative of the presence of, and/or increase in amount of, the target nucleic acid sequence.

Relatedly, the present invention also includes a reaction mixture, comprising a sample containing one or more target nucleic acids of interest, amplification reagents for amplifying the target nucleic acid sequence(es) of interest, and at least a first probe set comprising a capture probe comprising a fluorophore attached thereto, and a target specific nucleic acid probe complementary to at least a portion of the capture probe and to at least a portion of a target nucleic acid sequence, and comprising a quencher attached thereto. The quencher is attached to the probe such that it quenches fluorescence from the fluorophore when the target specific probe is hybridized to the capture probe.

The invention also includes a reaction chamber, which comprises a reaction region having disposed therein a sample containing one or more target nucleic acid(s) of interest, amplification reagents for amplifying the target nucleic acid sequence(es) of interest, and at least a first probe set comprising a capture probe comprising a fluorophore attached thereto, and a target specific nucleic acid probe complementary to at least a portion of the capture probe and to at least a portion of a target nucleic acid sequence, and comprising a quencher attached thereto, such that the quencher quenches fluorescence from the fluorophore when the target specific probe is hybridized to the capture probe.

The invention also includes amplification primers and target probes to be used in embodiments of the methods/devices of the invention. For example, the invention includes primers and probes for use in the specific detection of variola virus (e.g., from samples that may comprise mixed poxvirus content). Such primers and probes include VAR-1 probe, VAR-2 probe, VAR-3 probe, VAR-4 probe, VAR-1 primer 1, VAR-1 primer 2, VAR-2 primer 1, VAR-2 primer 2; VAR-3 primer 1, VAR-3 primer 2, VAR-4 primer 1, and VAR-4 primer 2

### BRIEF DESCRIPTION OF THE DRAWINGS

FIG. 1 shows a schematic illustration of the assay methods of the invention

FIG. 2 provides a schematic illustration of a reaction/detection chamber device for use in carrying out amplification and detection reactions in accordance with the invention.

FIG. 3 shows a schematic illustration of an exemplary fluorescence detection system.

FIG. 4 shows a schematic illustration of an alternate mobile phase assay system for carrying out amplification and detection reactions in accordance with the invention.

FIG. 5 shows the results of amplification of 10,000 copies of MS2 target in the presence of all of the primers and probes specific for 10 different targets.

FIG. 6 shows the results of a titration of MS2 target concentration using an assay of the invention.

FIG. 7 shows the results of amplification of 1 million copies of FluA/H3 target nucleic aid sequence in the presence of labeled target specific probe sequences and unlabeled capture probes.

### DETAILED DESCRIPTION OF THE INVENTION

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. Thus, for example, reference to a “surface” e.g., of the consumable chamber discussed herein, optionally includes a combination of two or more surfaces, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology is used in accordance with the definitions set out below.

The present invention is generally directed to methods and systems for detection of target nucleic acids in sample materials using real time PCR based detection methods. The invention benefits from an ability to provide greater multiplex and reduced signal background levels over prior described methods.

An elegant approach to the multiplex issue noted above is presented in commonly owned U.S. patent application Ser. No. 13/399,872, which is incorporated herein by reference in its entirety for all purposes. In brief, in such approach a probe is provided with a first portion that is complementary to a target sequence, and a second labeled flap portion that is not complementary to the target sequence. The labeled flap portion is released upon amplification of the target sequence and is captured by a complementary capture probe sequence provided upon a solid support, e.g., a substrate surface. Accumulation of the labeled flap portion at the surface of the solid support indicates that the target sequence is present and is being amplified. By using different flap portion sequences for different target sequences being assayed for, and by arraying different capture probes at different locations on a substrate that are complementary to those flap portion sequences, one can effectively detect the presence of multiple different target sequences in a single sample through a single amplification reaction process. Furthermore, because the labeled flap portion does not need to hybridize to the target, its sequence can be selected based upon the desired capture probe sequence or sequences on the substrate. As a result, a universal capture probe, or set of capture probes can be used to assay for any target sequence or sequences.

The present invention provides further improvements on these methods that result in improved signal generation and reduced background signal levels. In particular, the methods of the present invention utilize a first set of target specific nucleic acid probes that are complementary to at least a portion of a target nucleic acid sequence of interest. The target specific probes also include a quencher group attached at a certain position of the target specific probe. The methods also employ a second set of nucleic acid capture probes that are complementary to the target specific probes. The capture probes also include a fluorophore attached to a position on the capture probe such that the fluorophore is quenched by the quencher group on the target specific probe when the two probes are hybridized together, and subjected to appropriate excitation illumination for the fluorophore. As a result, the fluorescent signal from the unhybridized capture probe will be substantially greater than that from the target specific probe-capture probe hybrid.

In the methods of the invention, a sample material that is being analyzed for the presence of a target nucleic acid sequence is amplified in the presence of the target specific nucleic acid probe and the labeled capture probe. As detailed further herein, the methods of the invention can also include detection of multiple target nucleic acid sequences within a sample through use of multiple target specific probes and spatially separated multiple capture probes, e.g., a probe array. As used herein, amplification refers to the iterative duplication of the target sequence of interest to increase the total number of target nucleic acid molecules. A number of different amplification processes are well known in the art, including the polymerase chain reaction (PCR), and the ligase chain reaction (LCR).

In particularly preferred aspects, the target sequences are amplified in a PCR reaction. Briefly, PCR amplification methods employ two primer sequences that are complemantary to and prime amplification of the anteparallel strands of a nucleic acid that includes the target sequence of interest, upstream of that target sequence, such that primer extension will replicate the nucleic acid sequences including the target sequence and its complement. The reaction involves repeated thermal cycling of the reaction mixture to melt apart the complementary strands of the target containing sequence, annealing of the primers to the separated strands, extension of those primers using a polymerase enzyme, and repeating these cycles to melt apart the copied strands and make further copies based upon these duplicate strands. As a result of the iterative duplication process, the target nucleic acid is duplicated in an exponential fashion, e.g., one target is copied to yield two copies, which are duplicated to make four copies, etc.

During the amplification process used in accordance with the invention, each amplification cycle reduces the amount of target specific probe molecules that are available to bind to and quench the labeled capture probes. The reduction in the available target specific probes may result from one or more of: sequestration of the target specific probes by the increasing number of target sequences in the amplifying sample, or the digestion of the target specific probes during the amplification process.

In the context of a real time PCR reaction, a sample containing a target sequence of interest is subjected to a PCR reaction that is configured to amplify the target sequence of interest, e.g., priming upstream sequences of both anteparallel strands of the target in the presence of both the target specific probe and the capture probe. For example, when using a polymerase enzyme with inherent exonuclease activity, any target specific probe that hybridizes to the target sequence during the amplification process, will be digested through this exonuclease activity. Over multiple cycles of amplification, the amount of target specific probe will be significantly decreased in the reaction mixture, and fewer of the fluorophores on the capture probes will be quenched, resulting in an increased fluorescent signal as the target sequence is amplified. By observing fluorescence from the reaction after one or more amplification cycles, one can identify where a target sequence is present. Likewise, as discussed in greater detail below, by observing the fluorescence increase over the various amplification cycles, one can even quantify the amount of target present in the starting material.

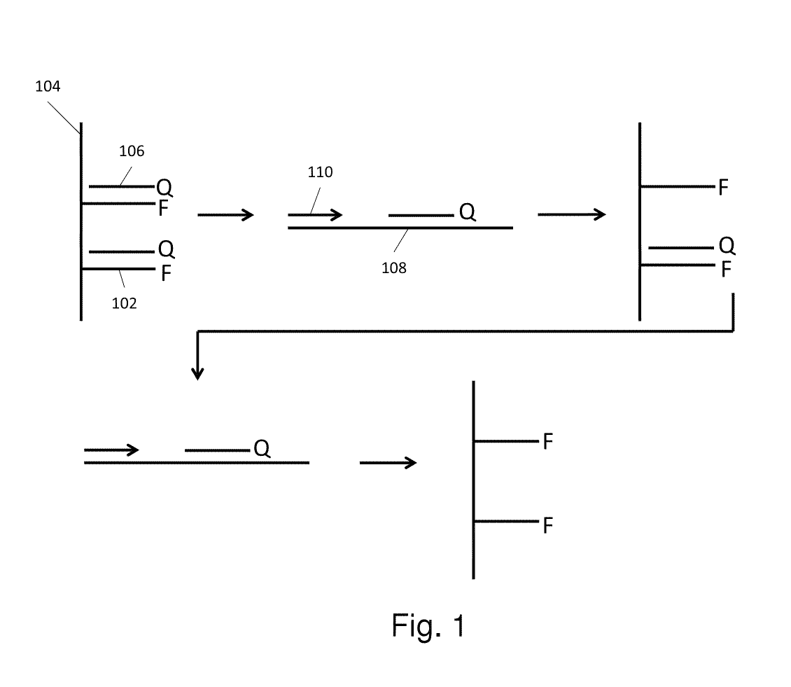

FIG. 1 schematically illustrates the reaction processes of the invention. As shown, set of capture probes 102, each of which probes bears an associated fluorescent moiety or fluorophore (F), is immobilized upon the surface of substrate 104. While the capture probes are preferably bound to a substrate surface for ease of interrogation and multiplexing, as described in greater detail below, this is not necessary for signal generation or detection under the methods of the invention. Target specific probes 106 are also provided that are complementary both to capture probes 102 and a target nucleic acid sequence of interest. These target specific probes include an associated quencher moiety (Q). The positioning of the fluorophore F on capture probe 102 and the quencher Q on target specific probe 106, are selected such that when probes 102 and 106 are hybridized together, the quencher Q is positioned sufficiently proximal to the fluorophore F as to quench its fluorescence when otherwise subjected to excitation illumination.

The above probes are then contacted with a sample material that is suspected of containing a target nucleic acid of interest, e.g., target sequence 108, and the target sequence is subjected to a PCR reaction process using a polymerase that includes, for example an inherent exonuclease activity. The PCR process includes multiple iterative melting, annealing and extension reaction steps resulting in extension of an appropriate primer 110 across the target sequence 108. During each annealing step, at least some of the target specific probes 106 will anneal to the target sequence 108. As that target sequence is replicated by the polymerase during the extension reactions, the target specific probes 106 that are hybridized to the target are digested by the exonuclease activity of the polymerase enzyme, preventing them from hybridizing with the capture probes 102, thus leaving the capture probes' associated fluorophores unquenched.

Although in preferred aspects, the methods of the invention employ a PCR reaction using a polymerase with inherent exonuclease activity, as noted above, such activity is not required for signal generation upon amplification of the target sequence of interest. In particular, an equilibrium will exist in a given reaction mixture for the target specific probe binding to either the capture probe or the target sequence. As the target sequence is amplified during the PCR reaction that equilibrium would shift toward more of the target specific probe binding to the target, rather than binding to and quenching the labeled capture probe. As a result, that amplification would result in an increase in fluorescent signal.

As will be appreciated, both for methods with and without exonuclease activity, the sensitivity of the reaction over the course of the amplification reaction will depend, at least in part, on the relative concentration of target specific probe and capture probe. For example, and as noted above, one would expect an equilibrium state of target specific probes being hybridized to any target present in the sample and to the capture probes, resulting in the possibility of some labeled capture probes being in an unhybridized, and unquenched state. Where the copy number of target sequences is low relative to the copy number of target specific probes, as will generally be the case, this would be expected to be an insubstantial effect. Typically, the amount of target produced during amplification will be so large as to allow starting target concentrations that are far below any measurable effect. In cases where target copy number is substantially higher, it could elevate an initial background fluorescence level as a result of sequestration of target specific probes. In such situations, it may be desirable to provide higher concentrations of target specific probes to counter this effect. Precise concentrations of the target specific probes may be adjusted based upon the copy number of target sequences to adjust for reduced baseline.

Alternatively, capture probe regions may be provided in two, three or more densities, or capture efficiencies, allowing capture to be optimally tuned to be sensitive to the concentration range spanning that concentration range from prior to following amplification cycles. Therefore, for any given amplification reaction, one could select the appropriate array region, e.g., having the optimized capture efficiency.

As noted above, in preferred aspects, the capture probes are provided immobilized upon a solid support. Immobilization of the capture probes provides a convenient mechanism for concentrating the signal associated with the capture probes, e.g., a surface which can be interrogated using standard fluorescence detection techniques. In addition, a plurality of different capture probes, i.e., having different nucleic acid sequences, may be arrayed upon a surface with each different capture probe sequence being provided in a discrete location, such that multiple different target sequences can be detected in a single reaction process using a single array. In particular, multiple different target specific probe sequences are contacted with a sample suspected of containing one or more different target sequences, in the presence of an array of capture probes, where each location in the array includes a capture probe complementary for a different target specific probe. Upon amplification, those target sequences that are present will result in digestion of their associated target specific probes which will, in turn, result in an unquenching of the associated fluorescently labeled capture probe. By identifying which capture probes are producing increased fluorescence, one can ascertain which of the target sequences are present in the sample. Exemplary processes for the preparation of capture probe arrays are described in, for example, U.S. patent application Ser. Nos. 13/399,872 and 61/600,569, the full disclosures of which are incorporated herein by reference in their entirety for all purposes.

In this context, the invention provides methods and associated devices, systems and consumables that permit highly multiplexed detection of nucleic acids of interest, e.g., for the detection of viruses, bacteria, plasmodium, fungi, or other pathogens in a biological sample. In preferred aspects, the consumable comprises a signal-optimized chamber having a high-efficiency thermo-stable nucleic acid detection array on an interior surface of the chamber. The array is configured to include up to about 100 or more different labeled capture probes. The methods include target specific probes that are complementary to the capture probes on the array and where those target specific probes include a quencher moiety, as described above. As noted, during amplification of a portion of a target nucleic acid of interest in the chamber, the quencher bearing target specific probes are digested and their ability to quench the fluorescence of the capture probes is substantially eliminated. The target specific probes may be provided in contact with the capture probes on the array prior to introduction of the sample to the array. Alternatively, the sample material may be mixed with the target specific probes prior to introduction to the array.

Accordingly, in a first aspect, methods of detecting a target nucleic acid are provided. This includes providing a detection chamber that has at least one high efficiency nucleic acid detection array on at least one surface of the chamber. As used herein, a “high-efficiency nucleic acid array” is an array of capture nucleic acid probes that efficiently hybridize to a target specific probe under hybridization conditions. In typical embodiments, the array is formatted on an inner surface of a reaction/detection chamber. The array can be formed by any conventional array technology, from spotting to chemical or photochemical synthesis on the surface. High efficiency is achieved by controlling the length of the region of the capture probe that recognizes the probe (shorter probes hybridize more efficiently than long probes, down to a minimum hybridization length for the hybridization conditions). Capture sites, the location on the capture probe that hybridizes to the target probe, can be made more efficient/available for hybridization with the target probe by including a linking sequence or structure between the capture site and the surface (thus formatting the capture sites at a selected distance from the surface, which can reduce surface effects on hybridization). For example, nucleic acid sequences or polyethylene glycol linkers (or both) can be used.

The capture nucleic acid probes are configured to capture relatively small probe nucleic acids, which also increases array efficiency. Detection of binding of the target specific probes to the array is carried out by observing a reduction in, or quenching of, the fluorescence of the capture probes on the array in the presence of the target specific probes that include quencher groups. Conversely, detection of amplification of a target sequence is accomplished by detecting an increase in fluorescence from the capture probes, as the target specific quenching probes are consumed by the amplification reaction and/or sequestered by the amplified target.

Preferably, this is carried out in a reaction or detection chamber that is configured to enhance the overall reaction and detection. A “reaction chamber” or “detection chamber” generally refers to a partly or fully enclosed structure in which a sample is analyzed or a target nucleic acid is detected. The chamber can be entirely closed, or can include ports or channels fluidly coupled to the chamber, e.g., for the delivery of reagents or reactants. The shape of the chamber can vary, depending, e.g., on the application and available system equipment. In some cases, the chamber may be configured to reduce background signal proximal to the array. A chamber may be “configured to reduce signal background proximal to the array” by dimensionally shaping it to reduce signal background, e.g., by including a narrow dimension (e.g., chamber depth) near the array (thereby reducing the amount of solution-generated signal proximal to the array), or when the chamber is otherwise configured to reduce background, e.g., by the use of coatings (e.g., optical coatings) or structures (e.g., baffles or other shaped structures proximal to the array). Typically, the chamber is configured to have a dimension (e.g., depth) proximal to the array, such that signal in solution is low enough to permit signal differences at the array to be detected. For example, in one embodiment, the chamber is less than about 1 mm deep above the array; desirably the chamber is less than about 500 μm in depth. Typically, the chamber is less than about 400 μm, less than about 300 μm, less than about 200 μm, or less than about 150 μm in depth above the array. In one example provided herein, the chamber is about 142 μm in depth. Such thinner chambers also have less thermal mass, and can be temperature cycled more rapidly and more efficiently than thicker chambers, which is useful in thermally cycled amplification methods.

In some embodiments, sample that has one or more copies of the target nucleic acid to be detected is loaded into the detection chamber. One or more amplification primers and a target specific probe including the quencher group, are hybridized to the one or more target nucleic acid copies. At least a portion of one or more of the target nucleic acid copies is amplified in an amplification primer dependent amplification reaction. The amplification reaction, when performed using a polymerase with exonucleae activity, results in cleavage of the target specific probe, e.g., due to nuclease activity of an amplification enzyme. This results in a reduction of the number of target specific probes available to bind to the capture probes on the array, and thus a reduction in overall quenching of the fluorophores on those capture probes, i.e., resulting in an increase in fluorescence that is detected from the array surface, which increase is indicative of the presence of the target nucleic acid in the sample.

As noted, thin reaction or detection chambers are preferred for their lower thermal mass and reduced fluid volumes that can contribute to background signals. In typical embodiments, the chamber is less than about 1 mm in depth or other dimension proximal to the array, more typically about 500 μm or less in at least one dimension proximal to the array, preferably less than about 250 μm or less, e.g., between about 10 μm and about 200 μm and in some embodiments the chamber is about 150 μm in a dimension proximal to the array. In one example herein, the chamber is about 142 μm in depth above the array. In another example herein, the chamber is about 100 μm in depth. The relevant chamber dimension depends on the signal detection path of the detection system, for example, where the signal is generated by passing light onto the array, where some of the light escapes through the array and into the fluid above the array, the relevant dimension is the depth of the chamber above the array.

For other assay formats described above, e.g., as described in U.S. application Ser. No. 13/399,872, arrays are typically provided with a non-rate limiting number of capture nucleic acids that hybridize to labeled probes, e.g., released labeled probe fragments. In such methods, it is desirable to not saturate the array with labeled probes. However, in the context of the present invention, the number of labeled capture probes is tailored so as to produce a measurable change in the number of target probes bound to the capture probes as a result of the amplification of the target sequence. Because of the substantial change in target concentration during amplification and the corresponding amount of target specific probe consumed in a typical amplification reaction, the change in amount of target probe bound to the labeled capture probe will yield a measurable effect. While the amount of target specific probe in a reaction mixture can vary widely, in particularly preferred aspects, the concentration of target specific probes in the reaction mixture will range from about 10 nM to about 1 uM, and preferably between about 50 nM and about 200 nM.

Typical capture probe array densities are between about 350 fmol/cm² or greater, e.g., about 2,000 fmol/cm² or greater, 2,500 fmol/cm² or greater, 3,000 fmol/cm² or greater, 4,000 fmol/cm² or greater, 4,500 fmol/cm² or greater, or 5,000 fmol/cm² or greater. The efficiency of the array is also a function of the length of the probe to be captured. Shorter probes typically display more efficient hybridization, although the probes do have to be long enough to bind at a given Tₘ during hybridization. Typical probes to be captured by the array are about 50 nucleotides in length or less; the arrays comprise sites that have corresponding complimentary capture nucleic acid sequences (nucleic acid sequences of the capture probes can optionally also include additional sequences, e.g., to space the complimentary area (the capture site) above the surface, e.g., to reduce surface effects). More typically, the probes and sequences of the capture site (not including any optional additional sequences used for spacing) are about 40 nucleotides or less in length, e.g., from about 20 to about 35 nucleotides in length.

In some cases, the capture probes of the array, and complementary target specific probes used in a given analysis are selected such that they provide a narrow range of Tm over all members of the array. In particular, to ensure optimal and consistent hybridization to the capture array, the capture probes in a given array will each have a Tm within about 10° C. of every other member of the array, and preferably, within about 7° C., 5° C., or 3° C. of every other probe in the array. Such a narrow Tₘ range allows for consistent hybridization and resulting signal generation across all members of the array. Because the target specific probes have a sequence dictated by the target sequence of interest, the ability to control the Tm for the hybrid will be reduced. However, in general, different target specific sequences within an overall target may be selected to accomplish a narrower Tm range.

As noted above, the position of the fluorophore on the capture probe and the quencher on the target specific probe is selected such that when the two probes are hybridized together, the quencher is positioned sufficiently proximal to the fluorophore to allow energy transfer from that fluorophore to the quencher, when subjected to excitation illumination. In certain aspects, this is accomplished by attaching the quencher and fluorophore to complementary bases on their respective probe sequences. Attachment of labeling groups and quenchers to nucleotides, and particularly to the nucleobase portions of nucleotides is well known in the art.

In particularly preferred aspects, the quencher group is provided at or near the 5′ position of the target specific probe, so that it does not interrupt cleavage/digestion during the amplification reaction.

The orientation of the capture probes on an array surface may also be varied. For example, the capture probe may be provided attached to an array surface at its 3′ or 5′ end, and the fluorophore may be provided more or less proximal to the surface of the array. In particularly preferred aspects, the capture probe is tethered to the surface via its 5′ end and bears its fluorophore proximal to that end, e.g., on its 3′ terminal nucleotide. As will be appreciated, attachment of probes to an array surface may be carried out through direct covalent attachment to the surface or a coating on that surface, or via intervening association molecules, e.g., through biotin-avidin linkages, nucleic acid hybridization couplings, e.g., using a separate attached nucleic acid that is complementary to a tag sequence present on the end of the capture probe, or the like. Again, exemplary surfaces, coatings and nucleic acid immobilization techniques are described in, e.g., U.S. Patent Application No. 61/600,569, which was previously incorporated herein in its entirety for all purposes.

Sample can be loaded into a chamber of the devices/systems of the invention by any of a variety of mechanisms, depending on the precise configuration of the consumable. In one convenient application, the sample is loaded through at least one port or fluidic channel in operable communication with the chamber. For example, ports can be fabricated in a top surface of the consumable, with the ports leading into the chamber. This provides for simplified loading, e.g., via a pipette or other fluid delivery device. Alternatively, fluidic or microfluidic channels, capillaries, or the like, can be used for sample delivery.

The methods of the invention can be used for detection of a nucleic acid of interest in a sample and/or quantification of the nucleic acid, e.g., in real time. Thus, in one aspect, the target nucleic acid is optionally amplified in a plurality of amplification cycles prior to detecting signal from the array, with the target nucleic acid portion additionally being amplified after signal detection, i.e., in the presence of additional copies of the target specific probe. After one or more amplification cycles, the target specific probe sequences remaining free in the reaction mixture, e.g., undigested, and unhybridized to the amplified target sequences, are allowed to hybridize to the capture probes on the array, and quench their associated fluorophores. The array is then assayed for the presence of fluorescence with the detected signal intensity being correlated to presence and/or quantity of the target nucleic acid present in the sample. Typically, the sample is amplified for more than 1 cycle before initial detection, to increase the level of signal by increasing the number of targets specific probes that are consumed by the amplification reaction or sequestered by the increased quantity of amplified target sequences. For example, the target nucleic acid can optionally be amplified for at least, e.g., 2, 3, 4, 5 or more amplification cycles prior to detecting signal from the array.

The labeled capture probe typically comprises a fluorescent or luminescent label, although other labels such as quantum dots can also be used. In one preferred embodiment, the label is a fluorescent dye. The signal produced by the capture probe is typically an optical signal. Similarly, the quencher groups on the target specific probes are selected to be an energy acceptor from the fluorophores used, e.g., operating as an acceptor of energy at the wavelength emitted by the fluorophore. As used herein, the quencher may be a non-radiative quencher, e.g., that accepts energy from the fluorophore without emitting energy on the form of light. Alternatively, the quencher may emit light energy, but at a wavelength that is filtered out by the detection system used in analyzing the fluorescence from the array. Thus, a quencher, merely refers to a group that accepts the fluorescent energy from the fluorophore and does not emit within the detected wavelength range of the detection system used to monitor the fluorescence at the emission wavelength of the fluorophore. In still other aspects, the quencher on the target specific sequence may include a radiative quencher, and the overall analytical instrument system may include an optics system that is able to differentially detect radiation emitted by the “quencher” and that emitted by the capture probe's fluorophore, in the absence of the “quencher” group on the target specific sequence. As a result, when the target specific sequences are bound to the array, and illuminated with an excitation wavelength targeted to excite the capture probe fluorophore, that fluorophore will emit at its first characteristic wavelength, which will be absorbed by the “radiative” quencher group via energy transfer. As a result, the radiative quencher will emit at its second characteristic wavelength. When the target specific probe is no longer bound to the array, e.g., as a result of consumption by the amplification reaction, the emitted energy from the unquenched capture probes' fluorophores will be emitted at the first characteristic wavelength. Using conventional dual wavelength fluorescence detection optics, one can detect both states of the array based upon their characteristic fluorescent signals.

Signal is typically detected by detecting one or more optical signal wavelengths corresponding to optical labels on the capture probes. Because binding position of target probes on the array can be used to discriminate between different target specific probes, it is not necessary to use different labels on the different probes to distinguish the probes in a multiplexed amplification reaction (an amplification reaction designed to amplify multiple target nucleic acids, if more than one of the targets is present in the sample). In other words, in some embodiments the position of the capture probe is specific for a given target sequence.

Although generally described in terms of label groups that are attached to the capture probe on the array, it will be appreciated that other detection schemes may be employed that do not require the use of pre-labeled capture probes. For example, in some embodiments, intercalating dyes may be used. Intercalating dyes typically provide a detectable signal event upon incorporation, or intercalation, into double stranded nucleic acids, e.g., the capture probe/target specific probe hybrid. In the context of the invention, the hybridization of the target specific probe to the complementary capture probe on the array creates a double stranded duplex at the array surface which could incorporate an intercalating dye, and provide a unique signal indicative of that hybridization. Intercalating dyes are well known in the art and include those described in, e.g., Gudnason et al., Nucleic Acids Research, (2007) Vol. 35, No. 19, e127, which is incorporated herein by reference for all purposes. Similarly, the target specific probe or probes may be provided with fluorescent or other optically detectable label groups, while the capture probes include no labeling groups or complementary groups, e.g., quenchers or energy acceptor groups. Prior to any amplification of the target sequences, these labeled probes will hybridize to their corresponding capture probes, creating a signal concentration at the surface of the substrate, e.g., the array. During amplification of the target sequence(s) in solution, the fluorescently labeled target specific probes are digested, releasing the label into the solution and reducing the amount of labeled target specific probe that hybridizes to the capture probes on the surface. As above, the presence of the target sequence in the sample results in a reduction in the amount of surface hybridized signal with successive amplification cycles. In some aspects, the target specific probes may be provided with included quencher groups, e.g., as in a conventional TaqMan® probe set.

Similarly, although optical signal detection methods are particularly preferred, the probe configurations and assay methods may also generally be practiced using non-optical labeling and/or detection methods, e.g., using electrochemical detection methods, e.g., ChemFETS, ISFETS, etc., optionally in conjunction which electrochemical labeling groups on the target specific probe, e.g., possessing large charged groups to amplify detection of hybridization of the target specific probe to an array probe at or near a detector surface. In the context of the invention, the signal associated with the target specific probe would thus decrease during the amplification process, as the target sequence is consumed or sequestered.

Local background can be detected for one or more regions of the array, with signal intensity measurements being normalized by correcting for said background. Typically, the normalized signal intensity is less than about 10% of total signal, e.g., between about 1% and about 10% of the total signal. In one example class of embodiments, the normalized signal intensity is between about 4% and about 7% of total signal. Typically, where approximately 1% or more of the signal is localized to the array, e.g., where about 1%, 2%, 3%, 4%, 5%, 6%, 7%, 8%, 9%, 10% or more of the signal is localized to the array for a region of the chamber, it is possible to discriminate the array signal from background. It is possible to discriminate even lower levels of signal to background, but this is not generally preferred. The methods can also include normalizing signal intensity by correcting for variability in array capture nucleic acid spotting (e.g., by correcting for spot size, spot density, or both), or by correcting for uneven field of view of different regions of the array.

The ability to simultaneously detect multiple target nucleic acids in a sample represents a preferred aspect of the invention. The sample may have one or a plurality of target nucleic acids, with the array comprising a plurality of capture nucleic acid types that are capable of detecting more than one target per sample. The capture nucleic acid types can be spatially separated on the array, eliminating the need for the use of multiple labels (although, as noted, multiple labels can be used). In multiplex approaches, a plurality of amplification probes, each specific for a different nucleic acid target, is incubated with the sample, which can include one or more target nucleic acids. For example, in some embodiments, there can be between about 5 and about 100 or more capture nucleic acid types. Each potential target to be detected will utilize a different target probe as well, e.g., there are optionally between about 5 and about 100 or more labeled target probe types in the amplification reaction, each specific for a potential target of interest. The array includes corresponding capture nucleic acid probes, e.g., between about 5 and about 100 or more capture nucleic acid probe types. This permits a corresponding number of signals to be detected and processed by the array. For example, between about 5 and about 100 or more different signals can be detected based upon positioning of the signals on the array after hybridization of the probe to the array. As will be appreciated, the number of capture probe types on an array will generally be dictated by the number of distinct amplification reactions that can be multiplexed within a single reaction volume. However, capture arrays having larger numbers of different capture probes, e.g., greater than 100, greater than 1000, 10,000 or more capture probe types, may also be employed in some circumstances, e.g., where amplification reactions are pooled for interrogation by the array, or the like.

While much of the discussion herein is directed to PCR based amplification, other amplification reactions can be substituted. For example, multienzyme systems involving cleavage reactions coupled to amplification reactions, such as those including the cleavage of scissile bonds (see, e.g., U.S. Pat. No. 5,011,769; U.S. Pat. No. 5,660,988; U.S. Pat. No. 5,403,711; U.S. Pat. No. 6,251,600) and forked nucleic acid structures (U.S. Pat. No. 7,361,467; U.S. Pat. No. 5,422,253; U.S. Pat. No. 7,122,364; U.S. Pat. No. 6,692,917) can be used. Helicase dependent amplification coupled to TaqMan® like cleavage (Tong, Y et al. 2008 BioTechniques 45:543-557) can also be used. Nucleic acid sequence based amplification (NASBA), or the ligase chain reaction (LCR) can be used. In NASBA-based approaches, the probe can be hybridized to a template along with amplification primer(s), as in PCR. The probe can be cleaved by the nuclease action of reverse transcriptase, or an added endonuclease, destroying or cleaving the probe in a manner similar to the release by a polymerase in PCR. One potential advantage of NASBA is that no thermocycling is required. This simplifies overall device and system requirements. For a description of NASBA, see, e.g., Compton (1991), “Nucleic acid sequence-based amplification,” Nature 350 (6313): 91-2. For the use of NASBA to detect, e.g., pathogenic nucleic acids, see, e.g., Keightley et al. (2005) “Real-time NASBA detection of SARS-associated coronavirus and comparison with real-time reverse transcription-PCR,” Journal of Medical Virology 77 (4): 602-8. When an LCR-style reaction is used, the probe can be cleaved using an endonuclease, rather than relying on nuclease activity of the amplification enzyme.

In typical embodiments, signals emanating from the array are detected and signal intensity is measured. Signal intensity is correlated to the presence and/or quantity of the target nucleic acid present in the sample. Typically, the sample is amplified for more than 1 cycle before initial detection, to increase the level of signal by increasing the number of target probes sequestered or digested by the amplification. For example, the target nucleic acid can optionally be amplified for at least, e.g., 2, 3, 4, 5, 6, 7, 8, 9, 10 or more amplification cycles prior to detecting signal from the array.

In one typical embodiment, fluorescent or other optical images are captured from the array at selected times, temperatures, and amplification cycle intervals, during the amplification reactions. These images are analyzed to determine whether the target nucleic acid(s) are present in the sample, and to provide quantification of starting target nucleic acid concentrations in the sample. The images are analyzed using a combination of mean gray intensity measurements, background correction and baseline adjustments. The background can be measured locally for each spot in the array. The background is computed by measuring the image intensity of a concentric annulus of the solution surrounding the array region (e.g., array spot) of interest. The signal from each region is then corrected to account for local background in the region. The corrected signal from each region can be further normalized to account for variability in spotting, as well as uneven illumination in the field of view. The average of the corrected intensity measurements obtained from the first few cycles, typically between cycle 5 and 15, are used to adjust the baseline and normalize measurements from each region.

Further details regarding methods of quantifying nucleic acids based upon signal intensity measurements following amplification can be found, e.g., in the references noted above and in Jang B. Rampal (Editor) (2010) Microarrays: Volume 2, Applications and Data Analysis (Methods in Molecular Biology) Humana Press; 2nd Edition ISBN-10: 1617378526, ISBN-13: 978-1617378522; Stephen A. Bustin (Editor) (2004) A-Z of Quantitative PCR (IUL Biotechnology, No. 5) (IUL Biotechnology Series) International University Line; 1st edition ISBN-10: 0963681788, ISBN-13: 978-0963681782; and Kamberova and Shah (2002) DNA Array Image Analysis: Nuts & Bolts (Nuts & Bolts series) DNA Press; 2nd edition ISBN-10: 0966402758, ISBN-13: 978-0966402759.

In some configurations, the capture probes may optionally be coupled to a mobile substrate, such as beads, resins, particles or the like (generally referred to interchangeably herein as “beads”), rather than a static substrate. For example, as noted elsewhere herein, a planar substrate may be used to provide arrayed capture probes that will hybridize with the target specific probes and their associated quenchers. The presence of a given target nucleic acid sequence is detected by detecting which capture probe position becomes unquenched through the digestion or sequestration of its complementary target specific probe. Because each capture probe and its complementary target specific probe is specific to a particular target sequence, if that capture probe is unquenched, it is indicative that the target was present and amplified.

In a mobile phase substrate, each different type of capture probe in a given analysis is coupled to a different mobile substrate that also bears a unique label. The mobile substrates are then passed through a detection channel in order to identify both the bead, and by implication, the capture probe, and whether the capture probe is quenched or unquenched. If the labeled capture probe is detected to be unquenched on a given bead that corresponds to a particular capture probe, it is indicative that the target sequence associated with that capture probe (and complementary target specific probe) was present in the sample and amplified. This aspect of the invention may be employed in endpoint detection, e.g., after completion of the overall amplification reaction, but may also be employed in quantitative analysis, e.g., siphoning a fraction of beads from the amplification mixture after one or more amplification cycles, and measuring the labeled probe signal intensity from the beads.

A variety of different bead types may be used in conjunction with this aspect of the invention. For example, polystyrene, cellulosic, acrylic, vinyl, silica, paramagnetic or other inorganic particles, or any of a variety of other bead types may be employed. As noted, the beads will typically be differentially labeled with a unique label signature. Again, a variety of different label types may be used, including organic fluorescent labels, inorganic fluorescent labels (e.g., quantum dots), luminescent labels, electrochemical labels, or the like. Such labels are widely commercially available and configured to be readily coupled to appropriately activated beads. In the case of fluorescent labeling groups, a large number of label signatures may be provided by providing different combinations of 2, 3, 4 or more spectrally distinct fluorescent labeling groups and different levels of each label, so as to provide a broad range of unique label signatures without having to use a broad spectrum of excitation radiation, e.g., multiple lasers.

The methods or the invention are typically performed using the devices, systems, consumables and kits herein. All features of the devices, systems and consumables can be provided to practice the methods herein, and the methods herein can be practiced in combination with the devices, systems, consumables and kits.

The reaction/detection chambers of the invention are typically employed in a one-pot format, e.g., reaction and detection occur in the same chamber. High-efficiency arrays are formed on at least one inner surface of the chambers. The arrays typically are in contact with amplification reactants and products during both amplification and array hybridization steps of the methods. This allows a user to run one or more amplification reaction cycles, detect the results by monitoring signal from the array in real time, and to then run one or more additional amplification cycles, again followed by detection. Thus, signal intensity from the array can be used to both detect and quantify one or more nucleic acids of interest, in real time.

The consumables of the invention include a chamber and a high efficiency array on an inner surface of the chamber. The chamber is typically thin (shallow), e.g., less than about 1 mm in depth. In general, the thinner the chamber, the less solution above the array, which reduces signal background from the solution. Typical desirable chamber depths are in the range of about 1 μm to about 500 μm. For ease of fabrication of the consumable, the chamber is often in the range of about 10 μm to about 250 μm in depth above the array, e.g., about 100 μm to about 150 μm in depth. The chamber can include a surface that has a reagent delivery port, e.g., for delivery of a sample by manual or automated pipettor.

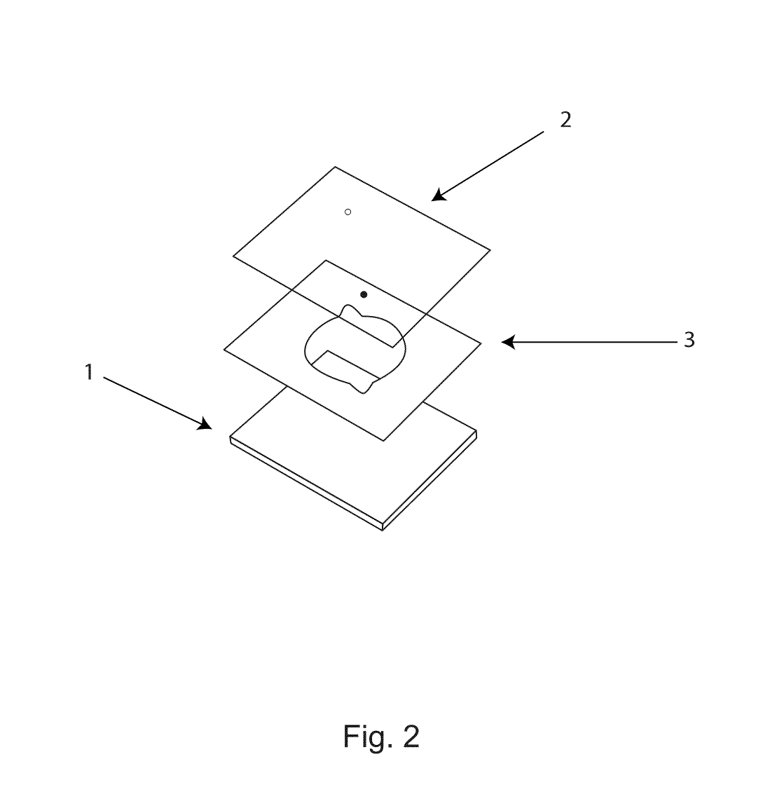

FIG. 2 provides a blow-up schematic of an example consumable. In this example, bottom surface layer 1 and upper surface layer 2, are joined by middle layer 3. Cutout 4 forms a chamber upon assembly of layers 1, 2, and 3. Port(s) 5 form(s) a convenient way to deliver buffer and reagents to the chamber upon assembly. A high efficiency array can be formed on the top or bottom layer in the region that forms the top or bottom surface of the cutout. In one convenient embodiment, where epifluorescent detection is used for detection of label bound to the array, the array is fabricated on the lower surface, with the consumable being configured to be viewed by detection optics located in the devices and systems of the invention below the lower surface. Generally, either the top or bottom surface (or both) will include a window through which detection optics can view the array.

Middle layer 3 can take any of a variety of forms, depending on the consumable assembly method to be used. In one convenient embodiment, top and bottom surfaces 1 and 2 are joined by layer 3 formed of a pressure-sensitive adhesive material. Pressure sensitive adhesive layers (e.g., tape) are well known and widely available. See, e.g., Benedek and Feldstein (Editors) (2008) Handbook of Pressure-Sensitive Adhesives and Products: Volume 1: Fundamentals of Pressure Sensitivity, Volume 2: Technology of Pressure-Sensitive Adhesives and Products, Volume 3: Applications of Pressure-Sensitive Products, CRC Press; 1st edition ISBN-10: 1420059343, ISBN-13: 978-1420059342.

Other fabrication methods for joining the top and bottom surface to form the chamber can also be used. For example, the top and bottom surfaces can be joined together by a gasket or shaped feature on the upper or lower surface, or both. The gasket or feature is optionally fused or adhered to a corresponding region of the upper or lower surface, or both. Silicon and polymer chip fabrication methods can be applied to form features in the top or bottom surface. For an introduction to feature fabrication methods, including micro-feature fabrication, see, e.g., Franssila (2010) Introduction to Microfabrication Wiley; 2nd edition ISBN-10: 0470749830, ISBN-13: 978-0470749838; Shen and Lin (2009) “Analysis of mold insert fabrication for the processing of microfluidic chip” Polymer Engineering and Science Publisher: Society of Plastics Engineers, Inc. Volume: 49 Issue: 1 Page: 104(11); Abgrall (2009) Nanofluidics ISBN-10: 159693350X, ISBN-13: 978-1596933507; Kaajakari (2009) Practical MEMS: Design of microsystems, accelerometers, gyroscopes, RF MEMS, optical MEMS, and microfluidic systems Small Gear Publishing ISBN-10: 0982299109, ISBN-13: 978-0982299104; Saliterman (2006) Fundamentals of BioMEMS and Medical Microdevices SPIE Publications ISBN-10: 0819459771, ISBN-13: 978-0819459770; and Madou (2002) Fundamentals of Microfabrication: The Science of Miniaturization, Second Edition CRC Press; ISBN-10: 0849308267, ISBN-13: 978-0849308260. These fabrication methods can be used to form essentially any feature that is desired on the top or bottom surface, eliminating the need for an intermediate layer. For example, a depression can be formed in the top or bottom surface (or both) and the two layers joined, thereby forming the chamber.

In some embodiments, the gasket or feature directs flow of a UV or radiation curable adhesive. This adhesive is flowed between the upper and lower surfaces and exposed to UV light or radiation (e.g., electron beam, or “EB” radiation), thereby joining the upper and lower surfaces. For a description of available adhesives, including UV and radiation curable adhesives, see, e.g., Ebnesajjad (2010) Handbook of Adhesives and Surface Preparation: Technology, Applications and Manufacturing William Andrew; 1st edition ISBN-10: 1437744613, ISBN-13: 978-1437744613; Drobny (2010) Radiation Technology for Polymers, Second Edition CRC Press; 2 edition ISBN-10: 1420094041, ISBN-13: 978-1420094046.

In other embodiments, the upper and lower surfaces can be ultrasonically fused together, with the gasket or surface feature delimiting regions that are fused and the chamber or other structural features to be produced in the consumable. Ultrasonic welding and related techniques useful for fusing materials are taught in, e.g., Astashev and Babitsky (2010) Ultrasonic Processes and Machines: Dynamics, Control and Applications (Foundations of Engineering Mechanics) Springer; 1st Edition. edition ISBN-10: 3642091245, ISBN-13: 978-3642091247; and Leaversuch (2002) “How to use those fancy ultrasonic welding controls,” Plastics Technology 48(10): 70-76.

In another example, the feature is a transparent region on either the upper or lower surface and a corresponding shaded region on a cognate upper or lower surface. In this embodiment, the upper and lower surfaces can be laser welded together by directing laser light through the transparent region and onto the shaded region. Laser welding methods are taught in, e.g., Steen et al. (2010) Laser Material Processing Springer; 4th ed. edition ISBN-10: 1849960615, ISBN-13: 978-1849960618; Kannatey-Asibu (2009) Principles of Laser Materials Processing (Wiley Series on Processing of Engineering Materials) Wiley ISBN-10: 0470177985, ISBN-13: 978-0470177983; and Duley (1998) Laser Welding Wiley-Interscience ISBN-10: 0471246794, ISBN-13: 978-0471246794.

In some embodiments, the capture nucleic acid array is typically coupled to a thermally stable coating on a window comprised as part of a consumable, detection chamber or the like. The window itself can include, e.g., glass, quartz, a ceramic, a polymer or other transparent material. A variety of coatings suitable for coating the window are available. In general, the coating is selected based upon compatibility with the array substrate (e.g., whether the chamber surface that the array is attached to is glass or a polymer), ability to be derivatized or treated to include reactive groups suitable for attaching array members, and compatibility with process conditions (e.g., thermostability, photostability, etc.). For example, the coating can include a chemically reactive group, an electrophilic group, an NHS ester, a tetra- or pentafluorophenyl ester, a mono- or dinitrophenyl ester, a thioester, an isocyanate, an isothiocyanate, an acyl azide, an epoxide, an aziridine, an aldehyde, an α,β-unsaturated ketone or amide comprising a vinyl ketone or a maleimide, an acyl halide, a sulfonyl halide, an imidate, a cyclic acid anhydride, a group active in a cycloaddition reaction, an alkene, a diene, an alkyne, an azide, or a combination thereof. For a description of surface coatings and their use in attaching biomolecules to surfaces see, e.g., Plackett (Editor) (2011) Biopolymers: New Materials for Sustainable Films and Coatings Wiley ISBN-10: 0470683414, ISBN-13: 978-0470683415; Niemeyer (Editor) (2010) Bioconjugation Protocols: Strategies and Methods (Methods in Molecular Biology) Humana Press; 1st Edition. edition ISBN-10: 1617373540, ISBN-13: 978-1617373541; Lahann (Editor) (2009) Click Chemistry for Biotechnology and Materials Science Wiley ISBN-10: 0470699701, ISBN-13: 978-0470699706; Hermanson (2008) Bioconjugate Techniques, Second Edition Academic Press; 2nd edition ISBN-10: 0123705010, ISBN-13: 978-0123705013. Wuts and Greene (2006) Greene's Protective Groups in Organic Synthesis Wiley-Interscience; 4th edition ISBN-10: 0471697540, # ISBN-13: 978-0471697541; Wittmann (Editor) (2006) Immobilisation of DNA on Chips II (Topics in Current Chemistry) Springer; 1st edition ISBN-10: 3540284362, ISBN-13: 978-3540284369; Licari (2003) Coating Materials for Electronic Applications: Polymers, Processing, Reliability, Testing (Materials and Processes for Electronic Applications) William Andrew ISBN-10: 0815514921, ISBN-13: 978-0815514923; Conk (2002) Fabrication Techniques for Micro-Optical Device Arrays Storming Media ISBN-10: 1423509641, ISBN-13: 978-1423509646; and Oil and Colour Chemists' Association (1993) Surface Coatings—Raw materials and their usage, Third Edition Springer; 3rd edition, ISBN-10: 0412552108, ISBN-13: 978-0412552106.

Methods of making nucleic acid arrays are available and can be adapted to the invention by forming the arrays on an inner chamber surface (e.g., of a consumable, detection chamber, etc.). Techniques for forming nucleic acid microarrays that can be used to form arrays on an inner chamber surface are described in, e.g., Rampal (Editor) Microarrays: Volume I: Synthesis Methods (Methods in Molecular Biology) Humana Press; 2nd Edition ISBN-10: 1617376639, ISBN-13: 978-1617376634; Muller and Nicolau (Editors) (2010) Microarray Technology and Its Applications (Biological and Medical Physics, Biomedical Engineering) Springer; 1st Edition. ISBN-10: 3642061826, ISBN-13: 978-3642061820; Xing and Cheng (Eds.) (2010) Biochips: Technology and Applications (Biological and Medical Physics, Biomedical Engineering) Springer; 1st Edition. ISBN-10: 3642055850, ISBN-13: 978-3642055850; Dill et al. (eds) (2010) Microarrays: Preparation, Microfluidics, Detection Methods, and Biological Applications (Integrated Analytical Systems) Springer ISBN-10: 1441924906, ISBN-13: 978-1441924902; Whittmann (2010) Immobilisation of DNA on Chips II (Topics in Current Chemistry) Springer; 1st Edition ISBN-10: 3642066666, ISBN-13: 978-3642066665; Rampal (2010) DNA Arrays: Methods and Protocols (Methods in Molecular Biology) Humana Press; 1st Edition ISBN-10: 1617372048, ISBN-13: 978-1617372049; Schena (Author, Editor) (2007) DNA Microarrays (Methods Express) Scion Publishing; 1st edition, ISBN-10: 1904842151, ISBN-13: 978-1904842156; Appasani (Editor) (2007) Bioarrays: From Basics to Diagnostics Humana Press; 1st edition ISBN-10: 1588294765, ISBN-13: 978-1588294760; and Ulrike Nuber (Editor) (2007) DNA Microarrays (Advanced Methods) Taylor & Francis ISBN-10: 0415358663, ISBN-13: 978-0415358668. Techniques for attaching DNA to a surface to form an array can include any of a variety of spotting methods, use of chemically reactive surfaces or coatings, light-directed synthesis, DNA printing techniques, and many other methods available in the art.

Methods of quantifying array densities are provided in the references noted above and in Gong et al. (2006) “Multi-technique Comparisons of Immobilized and Hybridized Oligonucleotide Surface Density on Commercial Amine-Reactive Microarray Slides” Anal. Chem. 78:2342-2351.

The consumable of the invention can be packaged in a container or packaging materials to form a kit. The kit can also include components useful in using the consumable, e.g., control reagents (e.g., a control template, control probe, control primers, etc.), buffers, or the like.

### Devices and Systems

Devices and systems that use the consumable and/or practice the methods of the invention are a feature of the invention as well. The device or system can include the features of the consumable, e.g., a reaction chamber and array (whether formatted as a consumable, or as dedicated portion of the device). Most typically, the device will typically have a receiver, e.g., a stage that mounts the consumable noted above, along with detection optics for monitoring the array, modules for thermocycling the chamber, and a computer with system instructions that control thermocycling, detection, and post-signal processing.

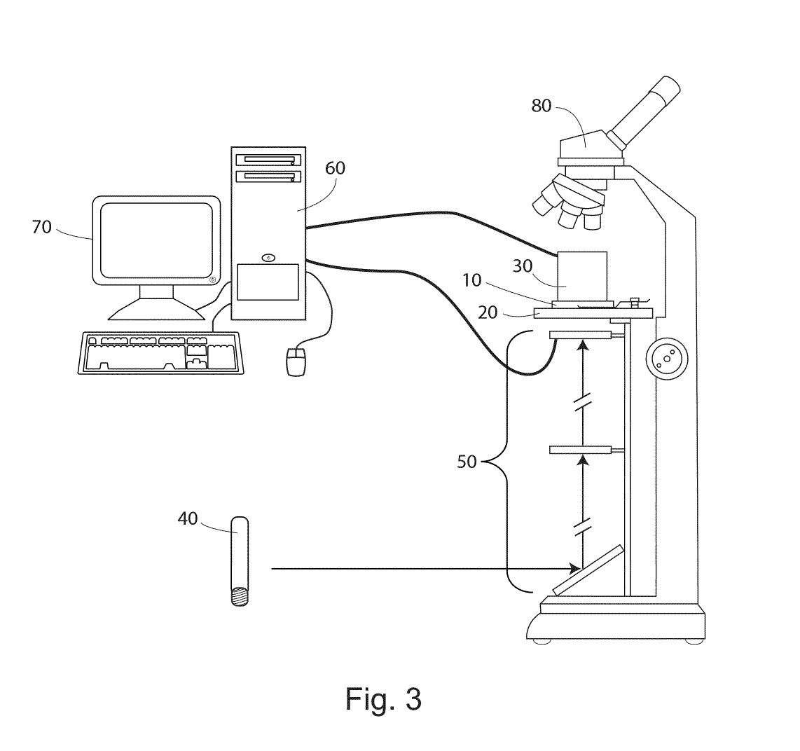

An example schematic system is illustrated in FIG. 3. As shown, consumable 10 is mounted on stage 20. Environmental control module (ECM) 30 (e.g., comprising a Peltier device, cooling fans, etc.) provides environmental control (e.g., thermocycling of temperature). Illumination light is provided by source 40 (e.g., a lamp, arc lamp, LED, laser, or the like). Optical train 50 directs light from illumination source 40 to consumable 10. Signals from consumable 10 are detected by the optical train and signal information is transmitted to computer 60. Computer 60 optionally also controls ECM 30 Signal information can be processed by computer 60, and outputted to user viewable display 70, or to a printer, or both. ECM 30 can be mounted above or below consumable 10 and additional viewing optics 80 (located above or below stage 20) can be included.

The stage/receiver is configured to mount the consumable for thermocycling and analysis. The stage can include registration and alignment features such as alignment arms, detents, holes, pegs, etc., that mate with corresponding features of the consumable. The stage can include a cassette that receives and orients the consumable, placing it in operable linkage with other device elements, although this is not necessary in many embodiments, e.g., where the consumable mounts directly to the stage. Device elements are configured to operate with the consumable and can include a fluidic delivery system for delivering buffers and reagents to the consumable, a thermocycling or other temperature control or environmental control module, detection optics, etc. In embodiments where the chamber is build into the device, rather being incorporated into the consumable, the device elements are typically configured to operate on or proximal to the chamber.

Fluid delivery to the consumable can be done by the device or system, or can be performed prior to loading the consumable into the device or system. Fluid handling elements can be integrated into the device or system, or can be formatted into a separate processing station discrete from the device or system. Fluid handling elements can include pipettors (manual or automated) that deliver reagents or buffers to ports in the consumable, or can include capillaries, microfabricated device channels, or the like. Manual and automated pipettors and pipettor systems that can be used to load the consumable are available from a variety of sources, including Thermo Scientific (USA), Eppendorf (Germany), Labtronics (Canada) and many others. Generally speaking, a variety of fluidic handling systems are available and can be incorporated into the devices and systems of the invention. See, e.g., Kirby (2010) Micro-and Nanoscale Fluid Mechanics: Transport in Microfluidic Devices ISBN-10: 0521119030, ISBN-13: 978-0521119030; Bruus (2007) Theoretical Microfluidics (Oxford Master Series in Physics) Oxford University Press, USA ISBN-10: 0199235090, ISBN-13: 978-0199235094; Nguyen (2006) Fundamentals And Applications of Microfluidics, Second Edition (Integrated Microsystems) ISBN-10: 1580539726, ISBN-13: 978-1580539722; Wells (2003) High Throughput Bioanalytical Sample Preparation: Methods and Automation Strategies (Progress in Pharmaceutical and Biomedical Analysis) Elsevier Science; 1st edition ISBN-10: 044451029X, ISBN-13: 978-0444510297. The consumable optionally comprises ports that are configured to mate with the delivery system, e.g., ports of an appropriate dimension for loading by a pipette or capillary delivery device.

The ECM or thermo-regulatory module can include features that facilitate thermocycling, such as a thermoelectric module, a Peltier device, a cooling fan, a heat sink, a metal plate configured to mate with a portion of an outer surface of the chamber, a fluid bath, etc. Many such thermo-regulatory components are available for incorporation into the devices and systems of the invention. See, for example, Kennedy and Oswald (Editors) (2011) PCR Troubleshooting and Optimization: The Essential Guide, Caister Academic Press ISBN-10: 1904455727; ISBN-13: 978-1904455721; Bustin (2009) The PCR Revolution: Basic Technologies and Applications Cambridge University Press; 1st edition ISBN-10: 0521882311, ISBN-13: 978-0521882316; Wittwer et al. (eds.) (2004) Rapid Cycle Real-Time PCR-Methods and Applications Springer; 1 edition, ISBN-10: 3540206299, ISBN-13: 978-3540206293; Goldsmid (2009) Introduction to Thermoelectricity (Springer Series in Materials Science) Springer; 1st edition, ISBN-10: 3642007155, ISBN-13: 978-3642007156; and Rowe (ed.) (2005) Thermoelectrics Handbook: Macro to Nano CRC Press; 1 edition, ISBN-10: 0849322642, ISBN-13: 978-0849322648. The thermo regulatory module can, e.g., be formatted into a cassette that receives the consumable, or can be mounted on the stage in operable proximity to the consumable.

Typically, the ECM or thermo regulatory module has a feedback enabled control system operably coupled to a computer which controls or is part of the module. Computer directed feedback enabled control is an available approach to instrument control. See, e.g., Tooley (2005) PC Based Instrumentation and Control, Third Edition, ISBN-10: 0750647167, ISBN-13: 978-0750647168; Dix et al. (2003) Human-Computer Interaction (3rd Edition) Prentice Hall, 3rd edition ISBN-10: 0130461091, ISBN-13: 978-0130461094. In general, system control is performed by a computer, which can use, e.g., a script file as an input to generate target temperatures and cycle time periods as well as to specify when images are to be viewed/taken by the detection optics. Photo images are typically taken at different times during a reaction and are analyzed by the computer to generate intensity curves as a function of time and thereby derive the concentration of the target.

The optical train can include any typical optical train components, or can be operably coupled to such components. The optical train directs illumination to the consumable, e.g., focused on an array of the consumable, or an array region. The optical train can also detect light (e.g., a fluorescent or luminescent signal) emitted from the array. For a description of available optical components, See, e.g., Kasap et al. (2009) Cambridge Illustrated Handbook of Optoelectronics and Photonics Cambridge University Press; 1st edition ISBN-10: 0521815967, ISBN-13: 978-0521815963; Bass et al. (2009) Handbook of Optics, Third Edition Volume I: Geometrical and Physical Optics, Polarized Light, Components and Instruments(set) McGraw-Hill Professional; 3rd edition, ISBN-10: 0071498893, ISBN-13: 978-0071498890; Bass et al. (2009) Handbook of Optics, Third Edition Volume II: Design, Fabrication and Testing, Sources and Detectors, Radiometry and Photometry McGraw-Hill Professional; 3rd edition ISBN-10: 0071498907, ISBN-13: 978-0071498906; Bass et al. (2009) Handbook of Optics, Third Edition Volume III: Vision and Vision Optics McGraw-Hill Professional, ISBN-10: 0071498915, ISBN-13: 978-0071498913; Bass et al. (2009) Handbook of Optics, Third Edition Volume IV: Optical Properties of Materials, Nonlinear Optics, Quantum Optics McGraw-Hill Professional, 3rd edition, ISBN-10: 0071498923, ISBN-13: 978-0071498920; Bass et al. (2009) Handbook of Optics, Third Edition Volume V: Atmospheric Optics, Modulators, Fiber Optics, X-Ray and Neutron Optics McGraw-Hill Professional; 3rd edition, ISBN-10: 0071633138, ISBN-13: 978-0071633130; and Gupta and Ballato (2006) The Handbook of Photonics, Second Edition, CRC Press, 2nd edition ISBN-10: 0849330955, ISBN-13: 978-0849330957. Typical optical train components include any of: an excitation light source, an arc lamp, a mercury arc lamp, an LED, a lens, an optical filter, a prism, a camera, a photodetector, a CMOS camera, and/or a CCD array. In one desirable embodiment, an epifluorescent detection system is used. The device can also include or be coupled to an array reader module, which correlates a position of the signal in the array to a nucleic acid to be detected.

In the context of the mobile substrate embodiments of the invention, in certain aspects, the reaction vessel may be coupled directly to a detection channel, e.g., within an integrated microfluidic channel system, or through an appropriate fluidic interface between the amplification mixture and the detection channel. Alternatively, a fluidic interface, such as is present in conventional flow cytometers, may be provided on the detection channel in order to sample the amplification reaction mixture. The detection channel is typically configured to have a dimension that permits substantially only single beads to traverse the channel at a given time. The detection channel will typically include a detection window allowing excitation of the beads and collection of the fluorescent signals emanating from the beads. In many cases, a fused silica or glass capillary or other transparent microfluidic channel is used as the detection channel.

The optical detection systems of the invention will typically include one or more excitation light sources capable of delivering excitation light at one or more excitation wavelengths. Also included will be an optical train that is configured to collect the light emanating from the detection channel, and filter excitation light from the fluorescent signals. The optical train also typically includes additional separation elements for transmitting the fluorescent signals, and for separating the fluorescent signal component(s) emanating from the bead and the signal component(s) emanating from the capture probe.

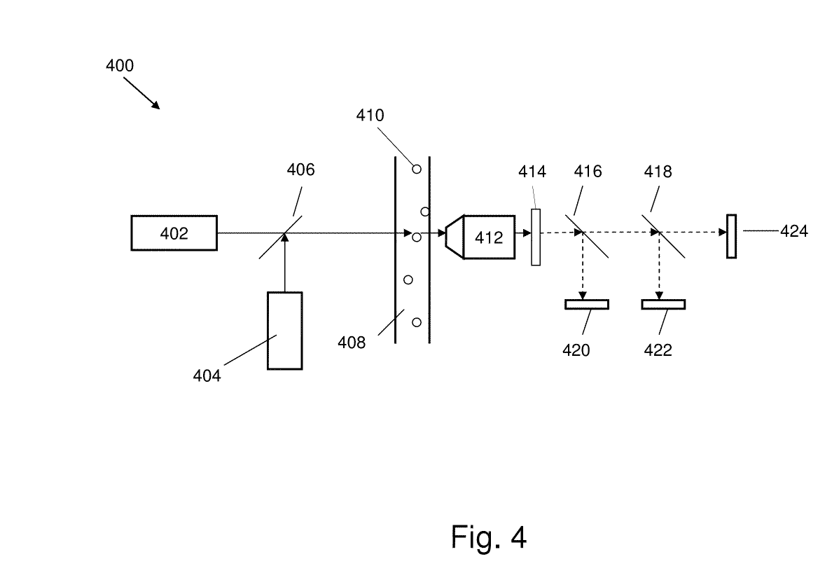

FIG. 4 provides a schematic illustration of an example overall detection system, system 400. As shown, the system includes first and second excitation light sources, such as lasers 402 and 404, that each provide excitation light at different wavelengths. Alternatively, a single broad spectrum light source or multiple narrow spectrum light sources may be used to deliver excitation light at the appropriate wavelength range or ranges to excite the detectable labels in the sample, e.g., those associated with the beads, and those associated with the labeled probe.

Excitation beams, shown as the solid arrows, from each laser are directed to detection channel 408, e.g., through the use of directional optics, such as dichroic 406. Light that emanates from beads 410 in detection channel 408, is collected by collection optics, e.g., objective lens 412. The collected light is then passed through filter 414 that is configured to pass the emitted fluorescence, shown as the dashed arrows, while rejecting the collected excitation radiation. The collected fluorescence includes fluorescence emitted from the label on the capture probe at a first emission spectrum, as well as fluorescent signals from the bead label signature, at one or more different emission spectra, depending upon the number of labels used in the beads. The collected fluorescence is then passed through dichroic 416 that reflects the fluorescence from the capture probe to first detector 420. The remaining fluorescent signature from the beads is then subjected to further separation by passing the signal through second dichroic 418, that reflects a first bead signal component to second detector 422, and passes a second bead signal component to third detector 424. The detectors are typically coupled to an appropriate processor or computer for storing signal data associated with detected beads, and analyzing the signal data to determine the identity of the bead, and thus the capture probe and associated target nucleic acid sequence. Additionally, the processor or computer may include programming to quantify signal data and originating target sequence copy number, where time course experiments are performed, e.g., beads are sampled after one or more amplification cycles in an overall amplification reaction.

The devices or systems of the invention can include, or be operably coupled to, system instructions, e.g., embodied in a computer or computer readable medium. The instructions can control any aspect of the device or system, e.g., to correlate one or more measurements of signal intensity and a number of amplification cycles performed by the thermo-regulatory module to determine a concentration of a target nucleic acid detected by the device.

A system can include a computer operably coupled to the other device components, e.g., through appropriate wiring, or through wireless connections. The computer can include, e.g., instructions that control thermocycling by the thermo-regulatory module, e.g., using feedback control as noted above, and/or that specifies when images are taken or viewed by the optical train. The computer can receive or convert image information into digital information and/or signal intensity curves as a function of time, determine concentration of a target nucleic acid analyzed by the device, and/or the like. The computer can include instructions for normalizing signal intensity to account for background, e.g., for detecting local background for one or more regions of the array, and for normalizing array signal intensity measurements by correcting for said background. Similarly, the computer can include instructions for normalizing signal intensity by correcting for variability in array capture nucleic acid spotting, uneven field of view of different regions of the array, or the like.

The methods of the invention were demonstrated experimentally as follows:

In one experiment, all primer and probe sequences were ordered from IDT (Coralville, Iowa) and received lyophilized. They were then resuspended in water to stock concentrations (100 uM to 200 uM) and used to prepare primer/probe stock solutions for PCR reactions. NVS PCR buffer was combined with primers and probes to make a PCR master mix.

Functionalized surfaces (COP substrates coated with functionalized polymer) were spotted using traditional microarray spotting techniques via an Array-It spotbot 2 (Sunnyvale, Calif.). Spotted slides were incubated at 75% humidity for 8-15 hours and then rinsed with DI water and dried with argon. Labeled capture probes were spotted in concentrations ranging from 100 nM to 50 uM in 50 mM pH 8.5 spotting buffer, producing a range of signal intensities.

Chip based reaction chambers were built on top of functionalized, arrayed surfaces using double sided pressure sensitive adhesive gaskets, polycarbonate lids and optically clear seals. Total volume of the reaction chamber was approximately 30 uL with a height of 150 um.

Target sequences of a known concentration were added to PCR master mix and the resulting solution was loaded in the reaction vessel. The vessel was sealed and loaded on a breadboard thermocycling instrument similar to that shown in FIG. 3.

Target sequences came from plasmid stocks or the resulting amplicons from previous reactions involving those amplicon stocks. All target molecules were quantitated using UV-Vis spectrometry on a NanoDrop 2000 instrument (ThermoScientific, Waltham, Mass.).

Thermocycling conditions included a hot start step at 95 C for 85 s, followed by 40 cycles of melt and extension of 5 s and 30 s respectively. Images of the surface were collected at the end of the extension steps. Average pixel intensity in the spots for each assay was calculated and plotted vs cycle number to generate real-time PCR curves. For 10-plex reactions, 250 nM probe/150 nM primer concentrations were used for all assays present.

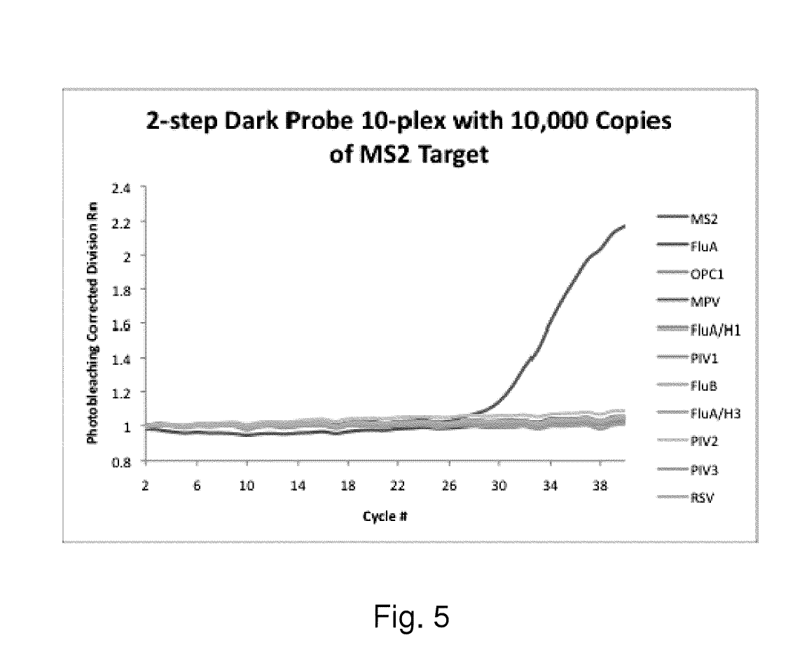

FIG. 5 shows the results of amplification of 10,000 copies of MS2 target in the presence of all of the primers and probes specific for 10 different targets.

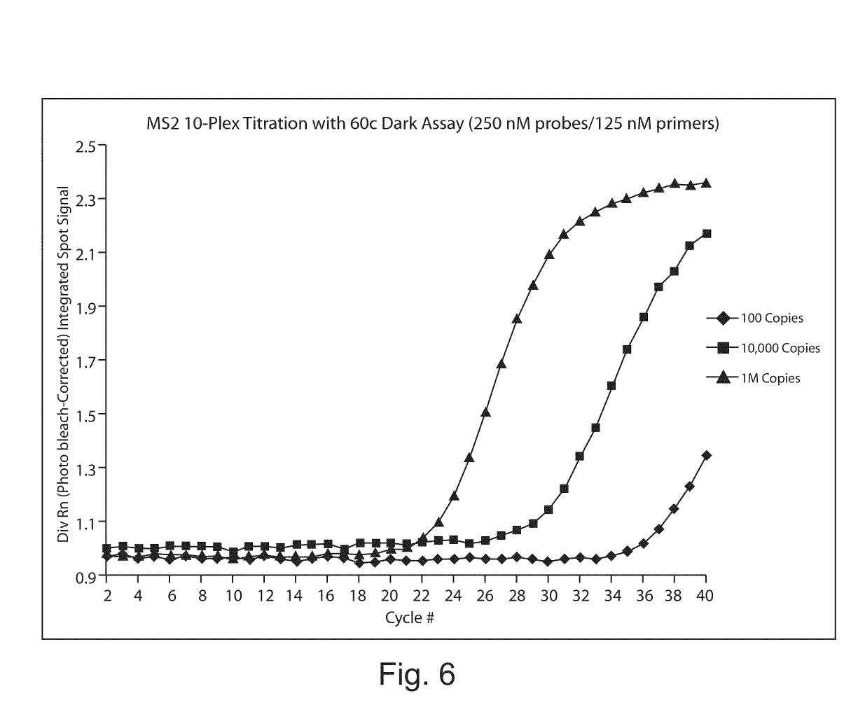

FIG. 6 shows the results of a titration of MS2 target concentration using an assay of this invention.

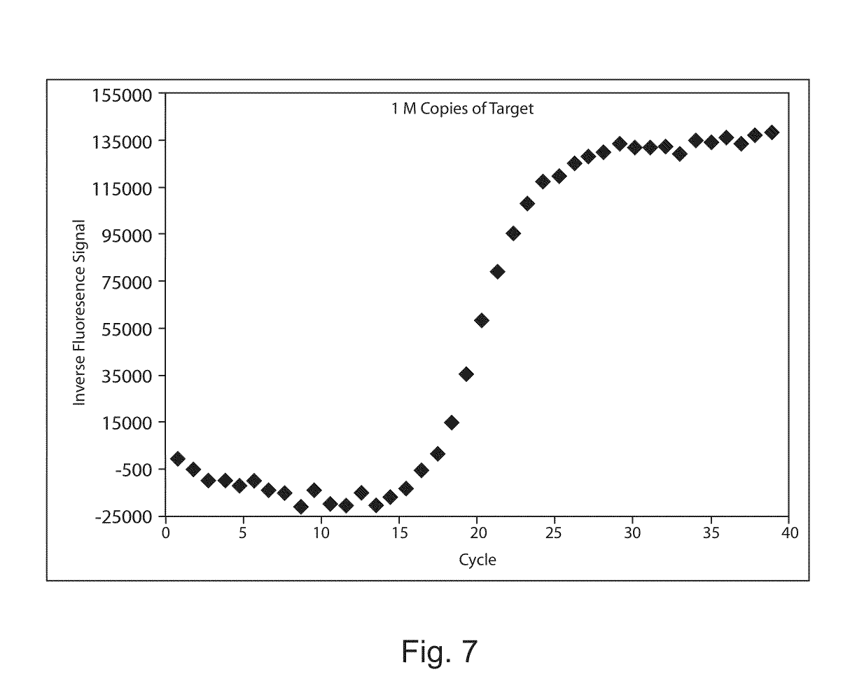

A similar experiment was performed using approximately 1 million copies of FluA/H3 target sequence. The amplification reaction was carried out in the presence of a standard Taqman® probe complementary to the target sequence in a reaction vessel that included an array spotted with unlabeled capture probes complementary to the Taqman probe. FIG. 7 shows a plot of inverse fluorescence at the array surface over multiple cycles of amplification. As can be seen in FIG. 7, the fluorescence decreases with increasing amplification cycles.

Another experiment illustrating aspects of the methods of the invention was directed towards assays of samples comprising poxvirus(es). In such experiment, various target probes and amplification primers were designed for use with the methods and devices of the invention. Variola virus, the causative agent of smallpox, is a member of the Poxvirus family and the Orthopoxvirus genus, which also includes the human pathogens vaccinia, cowpox, monkeypox, and several other related mammalian viruses. Variola virus can take two forms—variola major, which causes a severe disease having a fatality rate of 30-40%, and variola minor, which has a fatality rate of less than 1%. See, e.g., Harrison, et al., PNAS 101:11178-92 (2004).

Several PCR-based assays to detect Variola virus or other members of the Orthopoxvirus genus have been described. However, many of these assays appear to lack the specificity or generality they claim to have. For example, the majority of such prior assays target the hemagglutinin (HA) gene (see, e.g., Ropp, S. L., et al. “PCR Strategy for Identification and Differentiation of Smallpox and Other Orthopoxviruses” Microbiology 33:2069-2076 (1995); Ibrahim, M. S. et al. “Real-Time PCR Assay To Detect Smallpox Virus” Journal of Clinical Microbiology 41:3835-3839 (2003); Aitichou, M. et al. “Dual-probe real-time PCR assay for detection of variola or other orthopoxviruses with dried reagents” Journal of Virological Methods 153:190-5 (2008); Kulesh, D. A. et al. “Smallpox and pan-Orthopox Virus Detection by Real-Time 3J-Minor Groove Binder TaqMan Assays on the Roche LightCycler and the Cepheid Smart Cycler Platforms” Journal of Clinical Microbiology 42:601-609 (2004); and He, J. et al. “Simultaneous Detection of CDC Category “A” DNA and RNA Bioterrorism Agents by Use of Multiplex PCR & RT-PCR Enzyme Hybridization Assays” Viruses 1:441-459 (2009).). Also, while the HA gene, utilized in prior assays, has been noted as having some divergence between poxviruses (see, e.g., Nitsche, A., et al. “Detection of Orthopoxvirus DNA by Real-Time PCR and Identification of Variola Virus DNA by Melting Analysis” Journal of Clinical Microbiology 42:1207-1213 (2004).), the small deletions and SNPs that might differentiate between specific viruses are often not conserved among Variola virus strains themselves, making this target a difficult one for assay design. See, e.g., US20040029105 which deals with detection of Variola virus using HA as a target for amplification. Assays that target other regions have also been described (see, e.g., Kulesh, D. A., supra; Shchelkunov, S. N., et al. “Multiplex PCR detection and species differentiation of orthopoxviruses pathogenic to humans” Molecular and Cellular Probes 19:1-8 (2005); Fedele, C. G., et al., “A Use of internally controlled real-time genome amplification for detection of variola virus and other orthopoxviruses infecting humans” Journal of Clinical Microbiology 44:4464-70 (2006); Scaramozzino, N. et al. “Real-time PCR to identify variola virus or other human pathogenic orthopox viruses” Clinical Chemistry 53:606-13 (2007); and Loveless, B. M. et al. “Differentiation of Variola major and Variola minor variants by MGB-Eclipse probe melt curves and genotyping analysis” Molecular and Cellular Probes 23:166-170 (2009).).

Genome sequences of one variola minor strain and many variola major strains are available in the NCBI nr database, as are genome sequences for many other members of the Poxvirus family, thus allowing for an in silico search for unique regions within their genomes. Even so, however, differential detection of these viruses through prior traditional assay formats can be difficult due to the high degree of genomic sequence similarity.

In the present experiment, the Insignia program (available online at the URL “insignia.cbcb.umd.edu”) was used to identify unique genomic regions of Variola virus (both major and minor included). These regions displayed enough divergence from other Poxviruses for use in design of nucleic acid-directed assays of the invention for specific detection of Variola virus (i.e. assays specific for Variola not giving false positive detection of vaccinia, cowpox, monkeypox, camelpox etc).

To identify unique regions of the Variola virus genome, the Insignia parameters were set to identify sequences included in all Variola major and minor genomes but not present in any other genomes (thereby excluding all other sequenced Poxviruses and all other sequenced organisms). The Insignia parameters for signature chain length, which indicates the length (in nucleotides) of the unique region were also set. For many organisms, setting a signature chain length to 100 nucleotides or greater often identifies multiple unique regions. In such instances, the entire region can be used as the target for a nucleic acid-directed assay (i.e. the forward amplification primer, the reverse amplification primer, and the target probe can all be composed of sequences unique to the target organism). For the present experiment, in the Variola-specific Insignia search, only signature chain lengths of ≦39 nucleotides generated unique regions. This is likely due to the high degree of sequence conservation among the poxviruses.

In the present experiment, when the signature chain length was set to ≧35 nucleotides, 14 regions were generated (Table 1; genomic numbering corresponds to Variola Major Virus Bangledesh-1975). These regions were put through a BLAST search against the NCBI nr database to check for uniqueness and to display the closest sequence matches. 12 of the 14 regions had only 1 to 3 nucleotides that were different from the closest hit sequence, which was likely to be not enough difference to generate Variola-specific assays, while two regions (Regions 7 and 8 in Table 1) displayed a greater degree of differentiation from the nearest matches. These two regions were therefore used to create assays as described further below.

Table 1 shows Insignia results for Variola virus (major and minor included)-specific regions. Signature chain length was set to ≧35 nucleotides.

TABLE 1

| | Starting | Stopping | |
|---|---|---|---|
| | Nucleotide | Nucleotide | |
| Region | Position | Position | Sequence |
| 1 | 153149 | 153183 | GCAAGGATATTCTCATGGAG |
| | | | ATATTAAAGCGAGCA |
| 2 | 6262 | 6297 | AGTATATGTTGGAGGACATT |
| | | | CGAGTTCATTGTTTTC |
| 3 | 128198 | 128233 | CGACTAAGTTCAAGTTCTCC |
| | | | TTGACAAGATCCATCT |
| 4 | 74961 | 74996 | CGGAATTGGAATGTTTTGGT |
| | | | CACTGGGGTAAAGTAA |
| 5 | 2634 | 2669 | ATTATGCGGTCCAGAGGGAA |
| | | | ATGGATATTGTTTTCA |
| 6 | 34950 | 34986 | TAACGTCTTTCCAAGTGGCA |
| | | | AATTCCAAATTTTTTTC |
| 7 | 970 | 1007 | AGTGTATTGAGAGTGAGTGA |
| | | | GCATGAAAAAGATTTAGT |
| 8 | 161598 | 161635 | AACACTATTAGGAGAAAGCC |
| | | | AGGTCATATGGAAGATGT |
| 9 | 184180 | 184218 | TGCTAGATTGTCCAGTGTGC |
| | | | CCCCAGGACAAGGTAAGGA |
| 10 | 23346 | 23384 | ATCTAGGTTTCAAAAGACTT |
| | | | GGCATATTAACCCAAGCAG |
| 11 | 14365 | 14403 | ATACGGCAGCAACTAGTATT |
| | | | ATCTCTACATTGTTTACGG |
| 12 | 48985 | 49023 | CTGATGGCGACATAATTGTC |
| | | | CAAAACTGCCAATCTATAA |
| 13 | 3808 | 3846 | TACCCAATTAGAACACGTAT |
| | | | GCTTATTATCATCATTCGG |
| 14 | 50495 | 50533 | CCGACACCATCAAAGAATTT |
| | | | TTATGATATAAGGAAACAG |

The first unique region of interest (Region 7 in Table 1) that was identified corresponds to nucleotides 970-1007 of Variola Major Virus Bangledesh-1975. Upon further analysis of the larger region surrounding this 38 nucleotide sequence, it was discovered that only the 31 nucleotide stretch from nucleotides 977-1007 was conserved within all sequenced Variola virus stains. Within that 31 nucleotide region, 5 nucleotides did not match the sequence of the closest neighbor, cowpox virus. This difference was utilized in assay VAR-1 (see Table 7 for amplification primer and target probe sequences) by placing Primer 1 in this sequence (see Table 2). Primer 2 of this assay took advantage of 2 additional SNPs, while the probe has exact homology with several poxviruses.

Table 2 shows the 120 base pair area around Insignia-identified Region 7 (which corresponds to the 119 base pair region of 977-1095 of Variola Major Virus Bangledesh-1975). The conserved 31 nucleotides within Region 7 are underlined. Amplification primers P1 and P2 and the target probe from VAR-1 are indicated in bold italics. SNPs between Variola virus and other genomes are highlighted in black boxes. SNPs within Variola virus (in >1 genome sequences) are indicated in gray boxes—and were, and typically should be, avoided in Variola assay designs.

TABLE 2

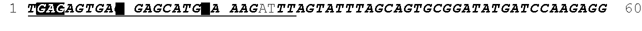

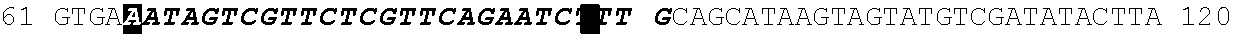

The second unique region identified (Region 8 in Table 1) corresponds to nucleotides 161598-161635 of Variola Major Virus Bangledesh-1975. When this sequence was subjected to a BLAST search against the NCBI nr database, 3 SNPs were identified in this region. More interestingly, when a larger region surrounding this sequence was analyzed, it was discovered that genomic rearrangements are found both upstream and downstream of this sequence in the most closely related genomes, those of some cowpox strains (see Table 3 below). These rearrangements allowed for the design of assays with amplification primers or target probes spanning regions that are contiguous in Variola virus but non-contiguous (junction regions) in other genomes. Such assays can afford even greater specificity than those that rely solely on SNPs for differentiation.

Table 3 shows the 300 base pair area around Insignia-identified Region 8. Region 8 is underlined. SNPs between Variola virus and other genomes are highlighted in black boxes. SNPs within Variola virus (in >1 genome sequences) are indicated in gray boxes—these were, and should be, avoided in Variola assay designs. The sequence indicated by # symbols underneath the sequence is duplicated in the nearest neighbor genomes (some cowpox genomes) and forms the upstream junction. The downstream junction (also present in the cowpox genomes) falls between the three nucleotides indicated by ̂ symbols underneath the sequence.

TABLE 3

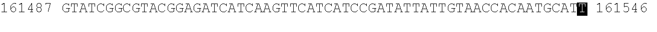

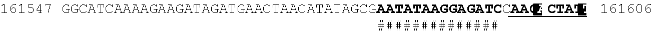

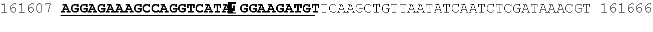

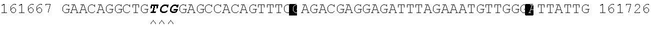

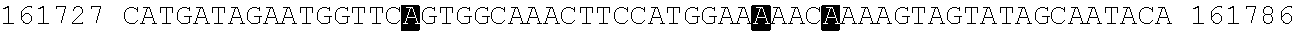

Three assays were designed to take advantage of these junction regions: VAR-2, VAR-3, and VAR-4 (again, see Table 7 for amplification primer and target probe sequences).

In the VAR-2 assay (Table 4), the P1 amplification primer spans the junction region just upstream of Insignia-identified Region 8, and the P2 amplification primer spans the junction region downstream of this region. The probe lies within the Insignia-identified Region 8 itself and takes advantage of the 3 SNPs in this region. Table 4 shows relative location variola-specific assay VAR-2. The amplification primer sequences P1 and P2 are indicated by asterisks underneath the sequences while the probe region is indicated by plus symbols underneath the sequence. Other formats are as in Table 3, except that the black-highlighted SNPs have been omitted for clarity.

TABLE 4

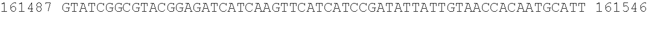

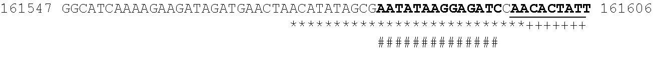

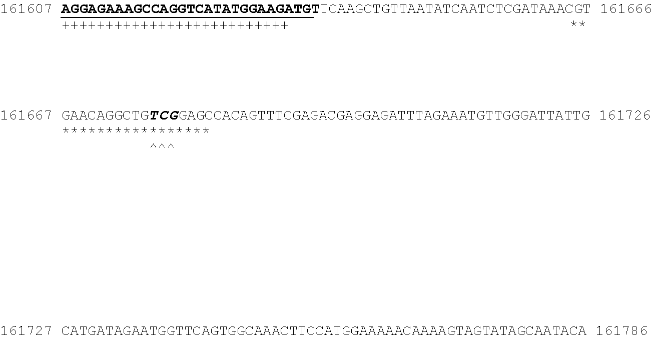

The VAR-3 assay (Table 5) has the probe spanning the upstream junction region, with the P1 and P2 amplification primers taking advantage of 1 SNP each as well as being on different sides of the junction (approximately 300 base pairs apart in the cowpox genomes, with the sequences inverted in those genomes). Table 5 shows variola-specific assay VAR-3. The P1 and P2 amplification primers are indicated by asterisks underneath the sequences while the probe region is indicated by plus symbols underneath the sequence. Other formats are as in Table 3, except that the black-highlighted SNPs have been omitted for clarity.

TABLE 5

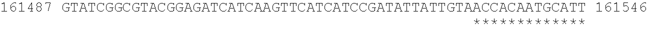

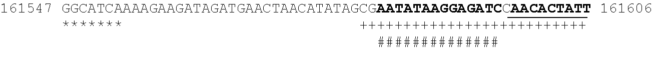

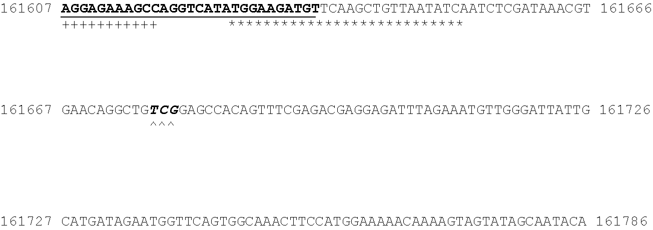

The sequence region amplified by the amplification primers (120 base pairs) in the VAR-4 assay (Table 6) spans the downstream junction, with P1 overlapping (in the opposite orientation) P2 from the VAR-3 region (118 bp), taking advantage of 1 SNP, the probe spanning the downstream junction, and P2 taking advantage of 1 SNP and being on the other side of the junction (about 7 kb away and inverted in near neighbors). Table 6 shows variola-specific assay VAR-4. P1 and P2 are indicated by asterisks underneath the sequences while the probe region is indicated by plus marks underneath the sequence. Other formats are as in Table 3, except that the black-highlighted SNPs have been omitted for clarity.

TABLE 6

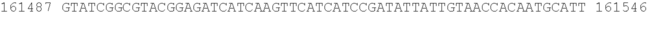

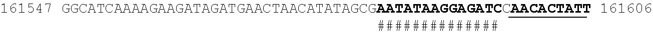

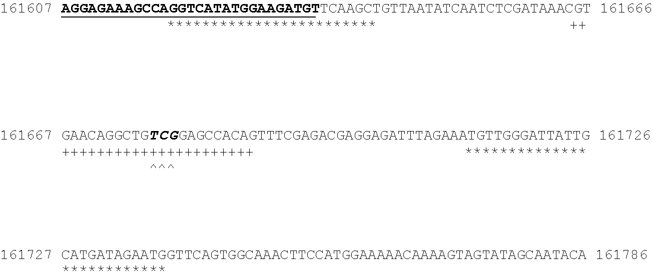

TABLE 7

| Assay | Amplicon | | P1 | | P2 | | Probe |
|---|---|---|---|---|---|---|---|
| Name | size | Primer 1 (P1) | length | Primer 2 (P2) | length | Probe | length |
| VAR-1 | 92 | TGA GAG TGA | 24 | CAA AAG ATT | 28 | TTA GTA TTT | 34 |
| | | GTG AGC ATG | | CTG AAC GAG | | AGC AGT GCG | |
| | | AAA AAG | | AAC GAC TAT T | | GAT ATG ATC | |
| | | | | | | CAA GAG G | |
| VAR-2 | 110 | ACA TAT AGC | 27 | GCT CCG ACA | 19 | TCT TCC ATA | 34 |
| | | GAA TAT AAG | | GCC TGT TCA C | | TGA CCT GGC | |
| | | GAG ATC CAA | | | | TTT CTC CTA | |
| | | | | | | ATA GTG | |
| VAR-3 | 118 | ACC ACA ATG | 20 | TGA TAT TAA | 27 | CGAATATAAGGAGA | 37 |
| | | CAT TGG CAT | | CAG CTT GAA | | TCCAACACTATTAG | |
| | | CA | | CAT CTT CCA | | GAGAAAGCC | |
| VAR-4 | 120 | GGTCATATGGAAG | 24 | CAT TCT ATC | 26 | CTG TGG CTC | 25 |
| | | ATGTTCAAGCT | | ATG CAA TAA | | CGA CAG CCT | |
| | | | | TCC CAA CA | | GTT CAC | |

Table 7 shows primer/probe sequences and characteristics of the Variola virus-specific assays.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A method of detecting the presence of at least a first target nucleic acid sequence in a sample, comprising:
subjecting the sample to an amplification reaction capable of amplifying the target nucleic acid sequence in the presence of at least a first set of nucleic acid probes, the first set of nucleic acid probes comprising:
a capture probe comprising a fluorophore attached thereto;
a target specific nucleic acid probe complementary to at least a portion of the capture probe and the target nucleic acid sequence, and comprising a quencher attached thereto, such that the quencher quenches fluorescence from the fluorophore when the target specific probe is hybridized to the capture probe; and
detecting fluorescence from the sample following one or more cycles of the polymerase chain reaction, an increase in fluorescence being indicative of the presence of the target nucleic acid sequence.

2. The method of claim 1, wherein the capture probe is attached to a surface of a substrate.

3. The method of claim 1, wherein the amplification reaction comprises a PCR reaction.

4. The method of claim 3, wherein the PCR reaction employs a polymerase enzyme having exonuclease activity.

5. The method of claim 4, wherein the PCR reaction is capable of amplifying a plurality of different target nucleic acid sequences and is carried out in the presence of a plurality of different sets of nucleic acid probes, each different set of probes comprising a different capture probe having a fluorophore attached thereto and a different target specific probe, each target specific probe being complementary to a different capture probe and a different one of the plurality of target nucleic acid sequences, and having a quencher attached thereto, such that the quencher quenches fluorescence from the fluorophore when each target specific probe is hybridized to a complementary capture probe.

6. The method of claim 5, wherein each of the capture probes in the plurality of different sets of probes is immobilized upon a substrate in a detectably distinct location.

7. The method of claim 1, wherein the at least first target nucleic acid sequence comprises a variola virus sequence.

8. The method of claim 7, wherein the target specific nucleic acid probe comprises one or more of: VAR-1 probe (SEQ ID NO: 19), VAR-2 probe (SEQ ID NO: 22), VAR-3 probe (SEQ ID NO: 25), or VAR-4 probe (SEQ ID NO:28).

9. The method of claim 1, wherein the target nucleic acid sequence is amplified through use of one or more of: VAR-1 primer 1 (SEQ ID NO: 17), VAR-1 primer 2 (SEQ ID NO: 18), VAR-2 primer 1 (SEQ ID NO: 20), VAR-2 primer 2 (SEQ ID NO: 21); VAR-3 primer 1 (SEQ ID NO: 23), VAR-3 primer 2 (SEQ ID NO: 24), VAR-4 primer 1 (SEQ ID NO: 26), or VAR-4 primer 2 (SEQ ID NO: 27).

10. A reaction mixture, comprising:
a sample containing a target nucleic acid of interest;
amplification reagents for amplifying the target nucleic acid sequence of interest; and
at least a first probe set comprising a capture probe comprising a fluorophore attached thereto, and a target specific nucleic acid probe complementary to at least a portion of the capture probe and the target nucleic acid sequence, and comprising a quencher attached thereto, such that the quencher quenches fluorescence from the fluorophore when the target specific probe is hybridized to the capture probe

11. The mixture of claim 10, wherein the target specific nucleic acid probe comprises one or more of: VAR-1 probe (SEQ ID NO: 19), VAR-2 probe (SEQ ID NO: 22), VAR-3 probe (SEQ ID NO: 25), or VAR-4 probe (SEQ ID NO: 28).

12. The mixture of claim 11, wherein the mixture further comprises one or more amplification primers chosen from the group consisting of: VAR-1 primer 1 (SEQ ID NO: 17), VAR-1 primer 2 (SEQ ID NO: 18), VAR-2 primer 1 (SEQ ID NO: 20), VAR-2 primer 2 (SEQ ID NO: 21); VAR-3 primer 1 (SEQ ID NO: 23), VAR-3 primer 2 (SEQ ID NO: 24), VAR-4 primer 1 (SEQ ID NO: 26), or VAR-4 primer 2 (SEQ ID NO: 27).

13. A reaction chamber, comprising:
a reaction region having disposed therein:
a sample containing a target nucleic acid of interest;
amplification reagents for amplifying the target nucleic acid sequence of interest; and
at least a first probe set comprising a capture probe comprising a fluorophore attached thereto, and a target specific nucleic acid probe complementary to at least a portion of the capture probe and the target nucleic acid sequence, and comprising a quencher attached thereto, such that the quencher quenches fluorescence from the fluorophore when the target specific probe is hybridized to the capture probe.

14. The reaction chamber of claim 13, wherein the capture probe is immobilized upon an interior surface of the reaction chamber.

15. The reaction chamber of claim 13, wherein a plurality of different capture probes are immobilized in an array on an interior surface of the reaction chamber, each different capture probe being immobilized in a discrete location.

16. A method of detecting the presence of at least a first target nucleic acid sequence in a sample, comprising:
subjecting the sample to an amplification reaction capable of amplifying the target nucleic acid sequence in the presence of at least a first set of nucleic acid probes, the first set of nucleic acid probes comprising:
a target specific nucleic acid probe having a fluorescent label associated therewith complementary to at least a portion of the target nucleic acid sequence, a capture probe associated with a solid support and complementary to at least a portion of the target specific nucleic acid probe; and
detecting fluorescence from the solid support following one or more cycles of the polymerase chain reaction, a decrease in fluorescence being indicative of the presence of the target nucleic acid sequence.

17. The method of claim 16, wherein the at least first target nucleic acid sequence comprises a variola virus sequence.

18. The method of claim 17, wherein the target specific nucleic acid probe comprises one or more of: VAR-1 probe (SEQ ID NO: 19), VAR-2 probe (SEQ ID NO: 22), VAR-3 probe (SEQ ID NO: 25), or VAR-4 probe (SEQ ID NO: 28).

19. The method of claim 16, wherein the target nucleic acid sequence is amplified through use of one or more of: VAR-1 primer 1 (SEQ ID NO: 17), VAR-1 primer 2 (SEQ ID NO: 18), VAR-2 primer 1 (SEQ ID NO: 20), VAR-2 primer 2 (SEQ ID NO: 21); VAR-3 primer 1 (SEQ ID NO: 23), VAR-3 primer 2 (SEQ ID NO: 24), VAR-4 primer 1 (SEQ ID NO: 26), or VAR-4 primer 2 (SEQ ID NO: 27).


SEQUENCE LISTING (28 sequences)

SEQ ID NO: 1 - DNA, 35 bp, Variola virus
gcaaggatattctcatggagatattaaagcgagca

SEQ ID NO: 2 - DNA, 36 bp, Variola virus
agtatatgttggaggacattcgagttcattgttttc

SEQ ID NO: 3 - DNA, 36 bp, Variola virus
cgactaagttcaagttctccttgacaagatccatct

SEQ ID NO: 4 - DNA, 36 bp, Variola virus
cggaattggaatgttttggtcactggggtaaagtaa

SEQ ID NO: 5 - DNA, 36 bp, Variola virus
attatgcggtccagagggaaatggatattgttttca

SEQ ID NO: 6 - DNA, 37 bp, Variola virus
taacgtctttccaagtggcaaattccaaatttttttc

SEQ ID NO: 7 - DNA, 38 bp, Variola virus
agtgtattgagagtgagtgagcatgaaaaagatttagt

SEQ ID NO: 8 - DNA, 38 bp, Variola virus
aacactattaggagaaagccaggtcatatggaagatgt

SEQ ID NO: 9 - DNA, 39 bp, Variola virus
tgctagattgtccagtgtgcccccaggacaaggtaagga

SEQ ID NO: 10 - DNA, 39 bp, Variola virus
atctaggtttcaaaagacttggcatattaacccaagcag

SEQ ID NO: 11 - DNA, 39 bp, Variola virus
atacggcagcaactagtattatctctacattgtttacgg

SEQ ID NO: 12 - DNA, 39 bp, Variola virus
ctgatggcgacataattgtccaaaactgccaatctataa

SEQ ID NO: 13 - DNA, 39 bp, Variola virus
tacccaattagaacacgtatgcttattatcatcattcgg

SEQ ID NO: 14 - DNA, 39 bp, Variola virus
ccgacaccatcaaagaatttttatgatataaggaaacag

SEQ ID NO: 15 - DNA, 120 bp, Variola virus
tgagagtgagtgagcatgaaaaagatttagtatttagcagtgcggatatgatccaagagggtgaaatagtcgttctcgttcagaatcttttgcagcataagtagtatgtcgatatactta

SEQ ID NO: 16 - DNA, 300 bp, Variola virus
gtatcggcgtacggagatcatcaagttcatcatccgatattattgtaaccacaatgcattggcatcaaaagaagatagatgaactaacatatagcgaatataaggagatccaacactattaggagaaagccaggtcatatggaagatgttcaagctgttaatatcaatctcgataaacgtgaacaggctgtcggagccacagtttcgagacgaggagatttagaaatgttgggattattgcatgatagaatggttcagtggcaaacttccatggaaaaacaaaagtagtatagcaataca

SEQ ID NO: 17 - DNA, 24 bp, Variola virus
tgagagtgagtgagcatgaaaaag

SEQ ID NO: 18 - DNA, 28 bp, Variola virus
caaaagattctgaacgagaacgactatt

SEQ ID NO: 19 - DNA, 34 bp, Variola virus
ttagtatttagcagtgcggatatgatccaagagg

SEQ ID NO: 20 - DNA, 27 bp, Variola virus
acatatagcgaatataaggagatccaa

SEQ ID NO: 21 - DNA, 19 bp, Variola virus
gctccgacagcctgttcac

SEQ ID NO: 22 - DNA, 33 bp, Variola virus
tcttccatatgacctggctttctcctaatagtg

SEQ ID NO: 23 - DNA, 20 bp, Variola virus
accacaatgcattggcatca

SEQ ID NO: 24 - DNA, 27 bp, Variola virus
tgatattaacagcttgaacatcttcca

SEQ ID NO: 25 - DNA, 37 bp, Variola virus
cgaatataaggagatccaacactattaggagaaagcc

SEQ ID NO: 26 - DNA, 24 bp, Variola virus
ggtcatatggaagatgttcaagct

SEQ ID NO: 27 - DNA, 26 bp, Variola virus
cattctatcatgcaataatcccaaca

SEQ ID NO: 28 - DNA, 24 bp, Variola virus
ctgtggctccgacagcctgttcac